SEQUENCE LISTING (6 sequences)

SEQ ID NO: 1 - protein, 1137 aa, Mucor ambiguus
MTKYAHDQIPGPEPHYLLGNVPDIFPDSLGNLIKLHDKYGPIVHLSMGGHELLSVDDPAVLETICEDNEYFTKEIESVYSDLAILNGRGLVTTSTADPDWQLGHKLIMNAFSARAMKAYHYKMGESISELCEIMDSFAKSGEDFDVSRWFIALALESIGKIGFDYDFDLLKDPNAPRHPFTVALAYVQSMIMKRASTLSWLKWYQTTTNVRFHRDLQTLRGTVEEVLKDRREHPHTEADQSDLLDFMIKAESKEGEKLNDSLIRDNIITFLSAGHNTTSAFLSWTMLELCKHPEVVENIKQEIANCGIKAGEVPTPEQVKECKYLDLVIKESLRIHPPITSILKYCKKDATVKASNGDEYDIKAGQLLQVNINALHHNPKVWEDPDVFNPDRFSGDTDNLPNTAWLTFSTGPRACIGRQFALQEGKLALVMILSRFHFKMDDPSQKIGYAVVVSTKPVGFFAKIESSQLPEPTEEIVVTKRRESKAVPQEKVKPAEFPLPPVTFLFGTQTNTSEEYARKLSGQAKEMGFKEVTVQDLDDWKLVKGEAIAKAQHDADAPSSEDDVKVSELVVVVTATYNGFPPDDANEFAKWLDERTKDSEATKNNMLSGMLYAVFGCGNRDWTSTFQKFPKKVDSGFELLGGERLLPAGEGDASDDIDGDFSLWSASFWTALMQRYGQSSSGKNADIMSNNGPAADPSQDFTLEFINIVKEKVKTEQAALNCNQLETVATIVENRELQHTEKSHRSTRHIQVQFDKSVDGKPLYEAGDHLEVVPVNEDRLVEIIATNLGLVLDSVFEVKDLDIKNLSPRSVAANIKGPCTIRNALKYYADLTGPPTRFSLSILSKQLKDSRPDIAERLQKALQPGKETERLKEFLASHRTLIDIIQAFKIKELNFKEFISSVNCIVPRKYSISSGPLEHPFDPSISVGVVDTVGGPDGNTHYFGLASGYLSHQEPGTKINAQIKACKSTFRLPDDPSTPVIFIAAGTGFSPFRGFLQERHAKGLKSSKKNSNGESSECYMFFGCRHPDQDFIYKEEFDAYLEDGTITELYTTFSRSGEVVKYVQHALLKHANLLYKLMEESNAKVYICGSAGSMAKDVKRTWERLLVQMSGVSESEAAEQIQAWVDEGKYNEDVWGT

SEQ ID NO: 2 - DNA, 3414 bp, Mucor ambiguus
  source 1..3414: mol_type = genomic DNA
atgaccaaatatgcccatgatcagattccgggtcctgaaccgcattatctgctgggtaatgttccggatatttttccggatagcctgggcaatctgattaaactgcatgataaatatggtccgattgtgcatctgagcatgggtggtcatgaactgctgagcgttgatgatccggcagttctggaaaccatttgtgaagataatgaatatttcaccaaagaaatcgaaagcgtgtatagcgatctggcaattctgaatggtcgtggtctggttaccaccagtaccgcagatccggattggcagctgggtcataaactgattatgaatgcatttagcgcacgtgccatgaaagcctatcactataaaatgggtgaaagcattagcgaactgtgcgaaattatggatagctttgcaaaaagcggtgaggattttgatgttagccgttggtttattgcactggcactggaaagcattggtaaaatcggtttcgattatgatttcgacctgctgaaagatccgaatgcaccgcgtcatccgtttaccgttgcgctggcctatgttcagagtatgatcatgaaacgtgcaagcaccctgagctggctgaaatggtatcagaccaccaccaatgttcgttttcatcgtgatctgcagaccctgcgtggcaccgttgaagaagttctgaaagaccgtcgtgaacatccgcataccgaagcagatcagagcgatctgctggattttatgattaaagccgaaagcaaagaaggcgagaaactgaacgatagcctgattcgtgataacatcattacctttctgagcgcaggtcataataccacctcagcatttctgagctggaccatgctggaactgtgtaaacatccggaagttgtcgaaaacatcaaacaagaaattgccaactgcggtattaaagcgggtgaagttccgacaccggaacaggttaaagaatgtaaatatctggacctggtgatcaaagaaagcctgcgtattcatccgcctattaccagcattctgaaatactgtaaaaaagacgcaaccgtgaaagccagcaatggtgatgaatatgatattaaagcaggtcagctgctgcaggttaacattaatgcactgcatcataacccgaaagtttgggaagatcctgatgtttttaacccggatcgttttagcggtgataccgataatctgccgaataccgcatggctgacctttagcaccggtccgcgtgcatgtattggtcgtcagtttgcactgcaagaaggtaaactggccctggttatgattctgagccgttttcatttcaaaatggatgatccgagccagaaaattggttatgcagttgttgttagcaccaaaccggttggtttttttgccaaaattgaaagcagccagctgccggaaccgaccgaagaaattgttgttaccaaacgtcgtgaaagtaaagcagttccgcaagaaaaagttaaaccggcagaatttccgctgcctccggtgacctttctgtttggcacccagaccaataccagcgaagaatatgcacgtaaactgagcggtcaggcaaaagaaatgggttttaaagaagttaccgtccaggatctggatgattggaaactggttaaaggtgaagcaattgcaaaagcacagcatgatgccgatgcaccgagcagcgaagatgatgttaaagtgagcgaactggttgttgttgtgaccgcaacctataatggttttccgcctgatgatgcaaacgaatttgcaaaatggctggatgaacgtaccaaagatagcgaagcaaccaaaaataacatgctgagcggtatgctgtatgcagtgtttggttgtggtaatcgtgattggaccagcacctttcagaaattcccgaaaaaagttgatagcggctttgaactgttaggtggtgaacgtctgctgccagccggtgaaggtgatgcaagtgatgatattgatggtgattttagcctgtggtctgccagcttttggaccgcactgatgcagcgttatggtcagagcagcagcggtaaaaatgcagatattatgagcaataatggtccggcagcagatccgagtcaggattttaccctggaatttatcaacatcgtgaaagagaaggtcaaaaccgaacaggcagcactgaattgtaatcagctggaaaccgttgcaaccattgttgaaaatcgcgaactgcagcatacagaaaaaagccatcgtagcacccgtcatattcaggttcagtttgataaaagcgtggatggtaaaccgctgtatgaagccggtgatcatctggaagtggttccggttaatgaagatcgtctggttgaaattattgccaccaatctgggtttagttctggatagcgtttttgaggtgaaagacctggatattaagaatctgagtccgcgtagcgttgcagcaaacattaaaggtccgtgtaccattcgtaatgccctgaaatattacgcagatctgaccggtcctccgacacgttttagtctgagcattctgtcaaaacagctgaaagacagccgtcctgatattgcagaacgcctgcagaaagcactgcagcctggtaaagaaaccgaacgtctgaaagaatttctggcaagtcatcgtaccctgatcgatattattcaggccttcaaaatcaaagaactgaacttcaaagagtttatcagcagcgttaattgcatcgttccgcgtaaatatagcattagcagtggtccgctggaacatccgtttgatccgagtattagcgttggtgttgttgataccgttggtggtccggatggtaatacccattattttggtctggcaagcggttatctgagccatcaagaaccgggtacaaaaatcaatgcacagattaaagcatgcaagagtacctttcgtctgccggatgatcctagcacaccggttatctttattgcagcaggcaccggttttagcccgtttcgtggttttctgcaagaacgtcatgcaaaaggtctgaaaagcagcaaaaaaaacagcaatggcgaaagcagcgagtgctatatgttttttggctgtcgtcatccggatcaggatttcatctataaagaagaatttgacgcctacctggaagatggcaccattacagaactgtataccacctttagccgtagcggtgaagttgttaaatatgttcagcacgcactgctgaaacatgcaaatctgctgtacaaactgatggaagaaagcaacgccaaagtgtatatttgtggtagcgcaggtagcatggcaaaagatgttaaacgtacctgggagcgcctgctggttcagatgagcggtgttagcgaaagcgaagcagcagagcagattcaggcatgggttgatgaaggcaaatataacgaagatgtttggggcacctaa

SEQ ID NO: 3 - protein, 1136 aa, Basidiobolus meristosporus
MSSQSDEIPGPKPHILLGNVQDIIPDTIGNHRRLHDEYGPVMRLYLGGHDFVSICDPEVIQSITGEGDFTKEIYSAYEDLAILSGRGLVTTATKDPDWILAHKLLMPAFSARAMKAYHDIMGKCILDLLKIMESYQECGEAIDMSRWMISLALESIGVIGFGYNFNLLDDKDSERHPFSVALNYVQSMIMKRTNSVTWTKWLQTTANVRFRRDLATLRDTVDDVLKQRRLNPPSEDARRDLLDFMLAAESKQGEKLDDNLIRDEIITFLSAGHNTTSSFLSWTFFELARNPDVEQKILQEIVNAGIKPGEIPSTEQVAKCKYIDMVIKESLRFHSPIPLVIKYCQNDCTIKTSEGKEYEIKRGQLAQIQISAVHKDPKLWENPNVFDPERFNPEKEADRHPCAWIPFSDGPRACIGRQFSLQEGKLALIMLLCKFRFRLLDETKEVGYQIIVSLKPVDLIMKVLPAELPSPNAGSDTSSVKDTVKPQLKPKENEVLEHARFPLPPVTFLYGTQTGTSEEYARKLSGQAKEFGFTDIAVAELDDWEVVKNNRIPSNKGQSSPSDEDGTKVSQLAVIVTATYNGYPPDNALKFDSWLTDITKDQKNQLEGLLYAVFGCGNKQWQSTFQAFPKKVDASIELLGAERLVPAGAGNADQDIDGDFTNWSASFWAALMQKYGRGASDKDADLMTTSGPVADPSNDFTLEFLPLGGDQAAMEAALGNRNSDPGAQVAILENKELQDVEKSGRSTRHIVVECPPASPGSGKKASYRAGDHLEIKPYNDDQLVENIAIGFGYVLDSVFQIKDCKITNLSPRSLAANIIGPCTVRNALTYFADLSGPPTRYTLTVMAKQLEKTRPDVAERLLHALQPGKETPRLREFLSTHRTILDIQRAFKIKELSFKEFLSSVNVIVPRRYSISSGPLEHPNEVSVTVGVVKDIGGADNNTDYYGLASGYLMRCPIGSKIDAKIKPCKNNFRLPENEQTPVIFICAGTGFAPFRGFLQERHAKGWKSKEKGGSSDAYLFFGCRHPDHDFIYQKELEEYLEDGTLTKLYTTFSRYNQTKKYVQHLLLTHAQLLFNLIMNENANIYVCGAGRGMAHDVRRTFERLAVQVAGMSEPEAVDAISHMVDAERYNEDVWG

SEQ ID NO: 4 - DNA, 3411 bp, Basidiobolus meristosporus
  source 1..3411: mol_type = genomic DNA
atgagcagccagagcgatgaaattccgggcccgaaaccgcatattctgctgggtaatgttcaggatattattccggataccattggcaatcatcgccgtctgcatgatgaatatggcccggttatgcgtctgtatctgggtggtcatgattttgtgagcatttgtgatccggaagtgattcagagcattaccggcgaaggcgattttaccaaagaaatctatagcgcatacgaagatctggcaattctgagcggccgtggcctggttaccaccgcaaccaaagatccggattggattctggcccataaactgctgatgccggcctttagtgcccgcgcaatgaaagcctatcatgatattatgggtaaatgtattctggatctgctgaaaattatggaaagctatcaggaatgtggtgaagccattgatatgagccgttggatgattagcctggcactggaaagtattggcgttattggttttggctataattttaatctgctggatgataaagacagcgaacgccatccgtttagcgttgcactgaattatgtgcagagtatgattatgaaacgcaccaatagcgttacctggaccaaatggctgcagaccaccgccaatgtgcgttttcgtcgcgatctggccaccctgcgcgataccgtggatgatgtgctgaaacagcgtcgtctgaatccgccgagcgaagatgcccgccgcgatctgctggattttatgctggccgccgaaagcaaacagggcgaaaaactggatgataatctgattcgcgatgaaattattacctttctgagtgcaggccataataccaccagtagttttctgagttggacctttttcgaactggcccgcaatccggatgtggaacagaaaattctgcaggaaattgtgaatgccggcattaagccgggtgaaattccgagtaccgaacaggtggccaaatgtaaatatattgatatggttatcaaggagagcctgcgctttcatagcccgattccgctggttattaagtattgtcagaatgattgcaccattaagaccagtgaaggtaaagaatatgaaattaagcgcggccagctggcccagattcagattagcgccgtgcataaagatccgaaactgtgggaaaatccgaatgtgtttgatccggaacgctttaatccggaaaaagaagcagatcgtcatccgtgtgcatggattccgtttagcgatggtccgcgtgcctgcattggtcgccagtttagcctgcaggaaggtaaactggccctgattatgctgctgtgcaaatttcgttttcgtctgctggatgaaaccaaagaagtgggttatcagattattgtgagcctgaaaccggtggatctgattatgaaagtgctgccggcagaactgccgagtccgaatgccggcagcgataccagtagtgtgaaagataccgttaaaccgcagctgaaaccgaaagaaaatgaagtgctggaacatgcccgttttccgctgccgccggttacctttctgtatggtacccagaccggcaccagtgaagaatatgcacgcaaactgagtggtcaggcaaaagaatttggctttaccgatattgccgtggcagaactggatgattgggaagttgttaaaaataatcgtatcccgagtaataagggtcagagtagtccgagcgatgaagatggcaccaaagtgagccagctggctgttattgttaccgcaacctataatggttatccgccggataatgcactgaaatttgatagctggctgaccgatattaccaaagatcagaaaaatcagctggaaggtctgctgtatgccgtgtttggttgtggcaataagcagtggcagagcacctttcaggcatttccgaaaaaagtggatgccagtattgaactgctgggcgcagaacgtctggtgccggcaggcgcaggcaatgccgatcaggatattgatggtgactttaccaattggagtgccagcttttgggccgcactgatgcagaaatatggccgcggcgccagtgataaagatgccgatctgatgaccaccagtggcccggtggccgatccgagtaatgattttaccctggaatttctgccgctgggtggtgaccaggcagcaatggaagcagccctgggtaatcgtaatagcgatccgggcgcacaggtggccattctggaaaataaggaactgcaggatgttgaaaaaagtggccgtagtacccgccatattgttgttgaatgcccgccggccagcccgggcagtggtaaaaaagcaagttatcgcgccggtgaccatctggaaattaagccgtataatgatgatcagctggtggaaaatattgccattggctttggctatgttctggatagtgtttttcagattaaggattgcaaaatcaccaatctgagtccgcgcagtctggccgccaatattattggtccgtgcaccgttcgcaatgccctgacctattttgccgatctgagtggtccgccgacccgctataccctgaccgtgatggcaaaacagctggaaaaaacccgcccggatgtggcagaacgtttactgcatgcactgcagccgggcaaagaaaccccgcgcctgcgcgaatttctgagtacccatcgtaccattctggatattcagcgcgcctttaaaattaaggaactgagctttaaagagttcctgagcagtgttaatgttattgttccgcgccgctatagtattagcagcggcccgctggaacatccgaatgaagtgagtgtgaccgtgggtgttgttaaagatattggcggtgcagataataataccgattattatggtctggccagtggttatctgatgcgctgcccgattggtagtaaaattgatgccaaaattaagccgtgcaaaaataattttcgcctgccggaaaatgaacagaccccggttatttttatttgcgccggcaccggctttgccccgtttcgtggttttctgcaggaacgccatgccaaaggttggaaaagtaaagaaaaaggcggtagtagcgatgcatatctgtttttcggctgtcgtcatccggatcatgattttatctatcagaaagaactggaagaatacctggaagatggtaccctgaccaaactgtataccacctttagccgctataatcagaccaaaaaatatgtgcagcatctgctgctgacccatgcccagctgctgtttaatctgattatgaatgaaaacgcaaacatctatgtgtgtggtgcaggccgtggcatggcccatgatgtgcgccgcacctttgaacgcctggcagttcaggttgcaggcatgagcgaaccggaagccgtggatgccattagtcacatggttgatgccgaacgctataatgaagatgtttggggttaa

SEQ ID NO: 5 - protein, 1135 aa, Umbelopsis isabellina
MTVYESDKIPGPEPRVLLGNIPDVYPDFVGNITQLHEKYGPVMRLYLGGHDYVSVCDPDCLQTTHKDGEYFTKEIQSTYEDLAILNGRGLVTTATKDPDWILGHKLLMPAFSARAMKAYHYKMGDTIKDLLNIIESFQKSGEDFDVSRWMIALALESIGNIGFDYDFNLLKDPNAERHPFTVALSYVQSMIMKRSASVSWMKWMKTSANARFQRDLGTLRNTLDSVLKERHEHPHSEDQQSDLLDFMISASTKEGDKLDDKIIRDNVMTFLSAGHNTTSSFLSWTILELCRHPEVAATIRQELANAGVQPGEIPTPEQVNACKYLDLVIKESLRMHPPIVAVLKYCKKDCTIKAGTTGDEYKIKAGQLLQSNINALHHSTKVWDEPMVFNPDRFADAELHPNAWMPFSDGPRACIGRQFSLQEGKLALVMMLSKFNFSMEDPSQKIGYEIIVAIKPVGLMVKVTPAELPEPTEEIVVTQRRESKAEPQESLKPAEFPLPPVTILYGTQTNTSEEYAKKLSGQAKEFGFKTIKVDDLDNWKLLNGGKLTKLNKDQSAPSSGDDVKVSELVVVVTATYNGNPPDNAMKFDEWLSKKTESIEDTKSNELEGILYAVFGCGNRDWSSTFQKFPTAVDTGLELLGGERLLPAGVGDASDDIDGDFSEWSANFWSTLMQRYGQSSSGKNADIMTSTAPLADPSKDFNLEFLPVHKNKELVTQANENRNQRGKTVTIKENRELQNIEKSHRSTKHIEVQFDKSEDGKPLYIAGDHLEITPVNKEELVELVAVNLGLVLDSVFQIQMNEVDISHLSPRSLAANIKGPCTIRNALKYYADLTGPPTRYTLSVLGKQLEKTRPEIAKRLQEALQPGKETPRLKEFLSTHRTFVDIMKAFNIKELNFKEFLSSVNCIVPRKYSISSGPTEHPFDPSVSIGIVRDIGGPDGKTEYRGLASGYLDTLKPGSQVNAQIKDCKSTFRLPDDGSTPVIFICAGTGMSPFRGFLQERHAQGLKSSKKGGSSEAYMFFGCRHPDQDFIYKDELQSYVEDGTLTELYTTFSRSNQVVKYVQHSLLQHAQMLYELMVDHNAKVYVCGSAGSMAKDVKRTWERITVQMSGMSEPEAEDLLKEWSDKGKYNEDVWGT

SEQ ID NO: 6 - DNA, 3408 bp, Umbelopsis isabellina
  source 1..3408: mol_type = genomic DNA
atgaccgtgtatgaaagcgataaaattccgggtccggaaccgcgcgttctgctgggtaatattccggatgtttatccggattttgttggcaatattacccagctgcatgaaaaatatggtccggttatgcgtctgtatctgggcggccatgattatgtgagtgtttgtgatccggattgtctgcagaccacccataaagatggtgaatattttaccaaagagatccagagcacctatgaagatctggccattctgaatggccgtggcctggtgaccaccgccaccaaagatccggattggattctgggccataaactgctgatgccggcattttcagcccgcgccatgaaagcctatcattataaaatgggtgacaccattaaggatctgctgaatattattgaaagttttcagaagtccggtgaagattttgatgttagccgttggatgattgccctggcactggaaagcattggcaatattggttttgattatgatttcaacctgctgaaagatccgaatgccgaacgtcatccgtttaccgttgccctgagttatgttcagagtatgattatgaaacgcagtgcaagcgttagctggatgaaatggatgaaaaccagtgccaatgcccgctttcagcgtgatctgggtaccctgcgcaataccctggatagtgtgctgaaagaacgccatgaacatccgcatagcgaagatcagcagagcgatctgctggattttatgattagtgccagtaccaaagaaggcgataaactggatgataaaattattcgcgataacgttatgacctttctgagcgcaggtcataataccaccagcagttttctgagttggaccattctggaactgtgccgtcatccggaagttgccgcaaccattcgtcaggaactggcaaatgccggtgtgcagccgggtgaaattccgaccccggaacaggtgaatgcctgtaaatatctggatctggttattaaggaaagcctgcgtatgcatccgccgattgtggcagttctgaaatattgtaaaaaggattgtaccatcaaggccggtaccaccggtgacgaatataaaattaaggcaggccagctgctgcagagcaatattaatgcactgcatcatagcaccaaagtttgggatgaaccgatggtttttaatccggatcgttttgccgatgcagaactgcatccgaatgcctggatgccgtttagtgatggtccgcgtgcctgtattggccgtcagtttagcctgcaggaaggcaaactggcactggttatgatgctgagcaaattcaattttagcatggaagatccgagccagaaaattggctatgaaattattgtggccattaagccggtgggcctgatggtgaaagtgaccccggccgaactgccggaaccgaccgaagaaattgttgttacccagcgccgtgaaagtaaagcagaaccgcaggaaagtctgaaaccggccgaatttccgctgccgccggttaccattctgtatggcacccagaccaataccagcgaagaatatgcaaaaaagctgagcggccaggccaaagaatttggttttaaaaccattaaggtggatgatctggataattggaaactgctgaatggcggtaaactgaccaaactgaataaggatcagagtgcaccgagtagtggtgacgatgtgaaagtgagtgaactggtggtggttgttaccgcaacctataatggcaatccgccggataatgcaatgaaatttgatgaatggctgagtaaaaagaccgaaagcattgaagataccaaaagcaatgaactggaaggcattctgtatgcagtttttggctgcggcaatcgtgattggagcagtacctttcagaaatttccgaccgcagtggataccggtctggaactgctgggcggcgaacgtctgctgccggcaggcgttggcgatgccagtgatgatattgatggcgattttagcgaatggagcgcaaatttttggagtaccctgatgcagcgttatggtcagagcagtagcggcaaaaatgcagatattatgaccagcaccgcaccgctggcagatccgagtaaagattttaatctggaatttctgccggtgcataaaaataaggaactggtgacccaggcaaatgaaaatcgcaatcagcgcggtaaaaccgtgaccattaaggaaaatcgcgaactgcagaatattgaaaaaagccatcgtagtaccaaacatattgaagttcagtttgataagagcgaagatggtaaaccgctgtatattgccggtgaccatctggaaattaccccggttaataaggaagaactggttgaactggtggccgttaatctgggtctggtgctggatagcgtgtttcagattcagatgaatgaagttgatattagtcacctgagcccgcgcagtctggcagcaaatattaagggcccgtgcaccattcgcaatgcactgaaatattatgcagatctgaccggtccgccgacccgttataccctgagtgtgctgggtaaacagctggaaaaaacccgtccggaaattgccaaacgtctgcaggaagccctgcagccgggcaaagaaaccccgcgcctgaaagaatttctgagtacccatcgtacctttgttgatattatgaaagcattcaatatcaaggagctgaattttaaagagttcctgagcagtgtgaattgtattgtgccgcgcaaatatagcattagtagtggtccgaccgaacatccgtttgatccgagcgtgagcattggtattgttcgcgatattggtggtccggatggcaaaaccgaatatcgcggtctggcaagcggctatctggataccctgaaaccgggtagtcaggtgaatgcgcagattaaggattgtaaaagtacctttcgcctgccggatgatggtagcaccccggttatttttatttgcgccggtaccggcatgagcccgtttcgtggctttctgcaggaacgccatgcccagggtctgaaaagcagtaaaaaaggtggcagtagtgaagcatatatgtttttcggttgccgccatccggatcaggattttatctataaagatgaactgcagagttacgttgaagatggtaccctgaccgaactgtataccacctttagtcgtagtaatcaggtggttaaatatgtgcagcatagcctgctgcagcatgcacagatgctgtatgaactgatggtggatcataatgcaaaagtttatgtttgtggcagcgccggtagcatggcaaaagatgttaaacgcacctgggaacgcattaccgttcagatgagtggcatgagcgaaccggaagccgaagatctgctgaaagaatggagcgataaaggcaaatataatgaagatgtgtggggcacctaa

(19) United States
(12) Patent Application Publication
(10) Pub. No.: US 2024/0076702 A1
(43) Pub. Date: Mar. 7, 2024

(54) BIOSYNTHETIC PRODUCTION OF GAMMA-LACTONES

(71) Applicant: Conagen Inc., Bedford, MA (US)

(72) Inventors: Hui Chen, North Billerica, MA (US); Oliver Yu, Lexington, MA (US)

(73) Assignee: Conagen Inc., Bedford, MA (US)

(21) Appl. No.: 18/062,628

(22) Filed: Dec. 7, 2022

Related U.S. Application Data
(62) Division of application No. 17/150,774, filed on Jan. 15, 2021, now Pat. No. 11,549,131.
Application No. 17/150,774 is a continuation of application No. PCT/US2019/042300, filed on Jul. 17, 2019.
(60) Provisional application No. 62/758,019, filed on Nov. 9, 2018.
(60) Provisional application No. 62/699,374, filed on Jul. 17, 2018.

(51) Int. Cl. C12P 17/04; C12N 15/52

(52) U.S. Cl. CPC C12P 17/04; C12N 15/52

(57) ABSTRACT

Provided herein are methods for making gamma lactones comprising reacting a carboxylic acid substrate with a heterologous cytochrome P450 (CYP450) protein with carboxylic acid 4-hydroxylase activity.

### RELATED APPLICATIONS

This application claims priority to and the benefit of U.S. Provisional Application No. 62/699,374, filed on Jul. 17, 2018, and U.S. Provisional Application No. 62/758,019, filed on Nov. 9, 2018, the disclosure of each of which is hereby incorporated by reference in its entirety.

### FIELD OF THE INVENTION

The field of the invention relates to methods and processes for producing gamma-lactones via recombinant proteins and/or microbial cultures modified to express such recombinant proteins. More specifically, the present disclosure relates to the production of C4-C20 gamma-lactones from corresponding carboxylic acid substrates via enzymatic conversion.

### BACKGROUND

There exists a universal desire for foods, fragrances and cosmetics that have pleasant tastes and odors. In many cases, these traits of pleasant smells and tastes are provided through various lactone compounds, including various gamma lactones, that possess desirable aroma and flavor characteristics. Current demand for lactone compounds is mainly addressed through either chemical synthesis or extraction processes from plants. Plant extraction-based production has significant disadvantages, such as weather effects on the strength and abundance of the compounds of interest, risk of plant diseases and/or poor harvest, stability of the compound, environmental impact of increased production and trade restrictions. Industrial production can cause environmental damage, may use dangerous precursors and itself may be subject to increased costs due to the costs of key substrates.

Accordingly, there is a need in the art for novel methods to produce gamma lactones economically and reliably without the limitations posed by plant extraction and chemical synthesis.

### SUMMARY OF THE INVENTION

According to the current invention, gamma-lactones may be reliably produced at high yield by gene modification and fermentation technology using microorganisms such as bacteria and/or yeasts. These microorganisms are able to synthesize lactones de novo or by biotransformation of fatty acids to provide commercially significant yields. Hence, new production methods are provided herein to reduce costs of gamma-lactone production and lessen the environmental impact of large-scale cultivation and processing of natural sources from which these lactone compounds can be extracted.

More specifically, the present disclosure encompasses methods and compositions for making gamma-lactones by microbial fermentation, wherein the microbial fermentation comprises a cellular system expressing a heterologous cytochrome P450 (CYP450) protein.

The present disclosure is based, in part, on the finding that certain CYP450 proteins and functional variants thereof have hydroxylase activity specifically at the carbon atom positioned gamma to the carbonyl carbon of a carboxylic acid substrate, thereby generating a 4-hydroxy carboxylic acid which upon acidification can convert into a gamma-lactone spontaneously. By overexpressing these CYP450 proteins in a modified microbial system and feeding into it the appropriate carboxylic acid substrates, various gamma-lactones can be produced at high titers. The present disclosure, therefore, provides economical and reliable methods for producing gamma-lactones from carboxylic acids (e.g., various commercially available fatty acids) without the disadvantages associated with chemical synthesis or plant extraction.

Accordingly, one aspect of the present disclosure provides a method for producing a C4-C20 gamma lactone comprising (a) incubating a cellular system expressing a heterologous CYP450 protein in a medium comprising a C4-C20 carboxylic acid substrate to provide a 4-hydroxy C4-C20 carboxylic acid, and (b) subjecting the 4-hydroxy C4-C20 carboxylic acid to acidic conditions to produce the C4-C20 gamma lactone. In some embodiments, the 4-hydroxy carboxylic acid can be isolated from the cellular system prior to acidification.

In various embodiments, the cellular system can be a transformed host cell comprising a sequence that encodes the heterologous CYP450 protein. In some embodiments, the cellular system can comprise bacterial cells, yeast cells, plant cells that do not naturally produce the lactone of interest, algal cells, and/or fungal cells that do not naturally encode the specified fungal CYP450 protein described herein. In particular embodiments, the cellular system can comprise transformed bacteria and/or yeast cells selected from the group consisting of Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveromyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Mucor; Pantoea; Corynebacterium; and Clostridium. In some embodiments, the cellular system can comprise growing cells. In other embodiments, the cellular system can comprise resting cells (e.g., lyophilized cells that have been frozen then re-suspended).

The present disclosure also encompasses a method for producing a C4-C20 gamma lactone, wherein the method involves providing a reaction mixture including a recombinant CYP450 protein described herein and a C4-C20 carboxylic acid substrate to provide a 4-hydroxy C4-C20 carboxylic acid, and subjecting the 4-hydroxy C4-C20 carboxylic acid to acidic conditions to produce the C4-C20 gamma lactone. In some embodiments, the recombinant CYP450 protein can be expressed by a transformed host cell in the reaction mixture. In other embodiments, the recombinant CYP450 protein can be isolated then provided in the reaction mixture.

In various embodiments of the methods for producing a C4-C20 gamma lactone according to the present disclosure, the CYP450 protein can be a Mucor ambiguous CYP450 protein or a functional variant thereof that has carboxylic acid 4-hydroxylase activity. For example, the CYP450 protein can comprise an amino acid sequence having at least 80%, 85%, 90%, 95%, 96%, 97%, 98%, or 99% identity to SEQ ID NO: 1. In certain embodiments, the CYP450 protein can comprise the amino acid sequence of SEQ ID NO: 1. In other embodiments, the CYP450 protein can consist of the amino acid sequence of SEQ ID NO: 1. Accordingly, the transformed host cell transformed to express such a CYP450 protein or variant thereof can comprise a nucleotide sequence having at least 80%, 85%, 90%, 95%, 97%, 98%, or 99% identity to SEQ ID NO: 2.

In some other embodiments, the CYP450 protein can be a Basidiobolus meristosporus CYP450 protein or a functional variant thereof that has carboxylic acid 4-hydroxylase activity. For example, the CYP450 protein can comprise an amino acid sequence having at least 80%, 85%, 90%, 95%, 96%, 97%, 98%, or 99% identity to SEQ ID NO: 3. In certain embodiments, the CYP450 protein can comprise the amino acid sequence of SEQ ID NO: 3. In other embodiments, the CYP450 protein can consist of the amino acid sequence of SEQ ID NO: 3. Accordingly, the transformed host cell transformed to express such a CYP450 protein or variant thereof can comprise a nucleotide sequence having at least 80%, 85%, 90%, 95%, 97%, 98%, or 99% identity to SEQ ID NO: 4.

In yet other embodiments, the CYP450 protein can be a Umbelopsis isabelline CYP450 protein or a functional variant thereof that has carboxylic acid 4-hydroxylase activity. For example, the CYP450 protein can comprise an amino acid sequence having at least 80%, 85%, 90%, 95%, 96%, 97%, 98%, or 99% identity to SEQ ID NO: 5. In certain embodiments, the CYP450 protein can comprise the amino acid sequence of SEQ ID NO: 5. In other embodiments, the CYP450 protein can consist of the amino acid sequence of SEQ ID NO: 5. Accordingly, the transformed host cell transformed to express such a CYP450 protein or variant thereof can comprise a nucleotide sequence having at least 80%, 85%, 90%, 95%, 97%, 98%, or 99% identity to SEQ ID NO: 6.

In some embodiments, the C4-C20 carboxylic acid substrate can include a C4-C20 carboxylic acid; a salt of a C4-C20 carboxylic acid; an ester of a C4-C20 carboxylic acid; a mono-, di- or triglyceride of a C4-C20 carboxylic acid; or a combination thereof. In certain embodiments, the C4-C20 carboxylic acid substrate can be a C4-C20 carboxylic acid or a salt thereof. The C4-C20 carboxylic acid can be a straight-chain C4-C20 carboxylic acid, a branched C4-C20 carboxylic acid, a saturated C4-C20 carboxylic acid, or an unsaturated C4-C20 carboxylic acid. The C4-C20 carboxylic acid can be optionally substituted with one or more functional groups selected from a hydroxyl group and an amino group, provided that such optional substitution is not at the carbon atoms that are positioned alpha, beta, or gamma to the carbonyl carbon of the carboxylic acid (see FIG. 1). In certain embodiments, the C4-C20 carboxylic acid can be a straight-chain fully saturated carboxylic acid. In particular embodiments, the C4-C20 carboxylic acid substrate can be selected from hexanoic acid, hexenoic acid, heptanoic acid, 1-heptenoic acid, 2-heptenoic acid, octanoic acid, 1-octenoic acid, 2-octenoic acid, 3-octenoic acid, nonanoic acid, 1-nonenoic acid, 2-nonenoic acid, 3-nonenoic acid, decanoic acid, 1-decenoic acid, 2-decenoic acid, 3-decenoic acid, undecanoic acid, 1-undecenoic acid, 2-undecenoic acid, 3-undecenoic acid, dodecanoic acid, 1-dodecenoic acid, 2-dodecenoic acid, 3-dodecenoic acid, tridecanoic acid, 1-tridecenoic acid, 2-tridecenoic acid, 3-tridecenoic acid, tetradecanoic acid, 1-tetradecenoic acid, 2-tetradecenoic acid, and 3-tetradecenoic acid.

Depending on the identity of the C4-C20 carboxylic acid substrate, the gamma lactones produced by the present methods can comprise a C4-C20 gamma lactone selected from γ-hexalactone, γ-hexenolactone, γ-heptalactone, γ-heptenolactone, γ-octalactone, γ-octenolactone, γ-nonalactone, γ-nonenolactone, γ-decalactone, γ-decacenolactone, γ-undecalactone, γ-undecenolactone, γ-dodecalactone, γ-dodecenolactone, γ-tridecalactone, γ-tridecenolactone, γ-tetradecalactone, and γ-tetradecenolactone.

Any methods described herein may further comprise isolating the gamma lactones from the cellular system to provide a crude product. In some embodiments, the crude product obtained from such isolating step can include a lactone content that is at least 70% pure. In some embodiments, the method further comprises purifying a crude product comprising the gamma lactones. In some embodiments, said crude product is purified by column chromatography. In some embodiments, said crude product is purified by acid-base extraction. In some embodiments, said crude product is purified by vacuum distillation. In some embodiments, the method further comprises purifying said lactones using a semi-preparative HPLC.

The gamma lactones produced by the methods described herein can be used alone or mixed with other lactones, flavors, or scents to obtain a desired composition for use in a variety of consumer products or foods. For example, the gamma lactones described herein can be included in food products (such as beverages, soft drinks, ice cream, dairy products, confectioneries, cereals, chewing gum, baked goods, etc.), dietary supplements, medical nutrition, as well as pharmaceutical products to give desired flavor or aromatic characteristics. In some embodiments, the disclosure provides a consumable product comprising a flavoring or scented amount of a lactone produced by any one of the methods above or described elsewhere herein for use in a specific flavoring context, formula or recipe. In some embodiments, the consumable product can be a food, beverage, perfume, or cosmetic product. In some embodiments, the composition can be selected from beverages, confectioneries, bakery products, cookies, and chewing gums. In certain embodiments, the composition can be a food, beverage, perfume, or cosmetic product having a flavor or aroma profile designed to taste or smell like: peach, apricot, pear, maple, coconut, vanilla, butterscotch, grenadine and/or date.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 compares the 4-hydroxylase route against the β-oxidation route for producing gamma-lactones from carboxylic acid substrates. Specifically, the production of γ-decalactone (GC10) is used as an example to illustrate the two routes.

FIGS. 2a-2h show GC/MS spectra confirming production of various γ-lactones using an E. coli culture transformed to overexpress Mucor ambiguus P450. Specifically, FIG. 2a shows the production of γ-heptalactone (GC7) from enanthic acid (heptanoic acid, C7). FIG. 2b shows the production of γ-octalactone (GC8) from caprylic acid (octanoic acid, C8). FIG. 2c shows the production of γ-nonalactone (GC9) from pelargonic acid (nonanoic acid, C9). FIG. 2d shows the production of γ-decalactone (GC10) from capric acid (decanoic acid, C10). FIG. 2e shows the production of γ-undecalactone (GC11) from undecylic acid (undecanoic acid, C11). FIG. 2f shows the production of γ-dodecalactone (GC12) from lauric acid (dodecanoic acid, C12). FIG. 2g shows the production of γ-tridecalactone (GC13) from tridecylic acid (tridecanoic acid, C13). FIG. 2h shows the production of γ-tetradecalactone (GC14) from myristic acid (tetradecanoic acid, C14).

FIG. 3 shows GC/MS spectra confirming γ-lactone production using an E. coli culture transformed to overexpress Basidiobolus meristosporus P450. Specifically, the top panel shows the production of γ-undecalactone (GC11) from undecylic acid (undecanoic acid, C11). The bottom panel shows the production of γ-undecenolactone (GC11′) from undecylenic acid (undecenoic acid, C11:1).

FIGS. 4a-4g show GC/MS spectra confirming production of various γ-lactones using an E. coli culture transformed to overexpress Umbelopsis isabellina P450. Specifically, FIG. 4a shows the production of γ-hexalactone (GC6) from hexanoic acid (C6). FIG. 4b shows the production of γ-heptalactone (GC7) from enanthic acid (heptanoic acid, C7). FIG. 4c shows the production of γ-decalactone (GC10) from capric acid (decanoic acid, C10). FIG. 4d shows the production of γ-undecalactone (GC11) from undecylic acid (undecanoic acid, C11). FIG. 4e shows the production of γ-dodecalactone (GC12) from lauric acid (dodecanoic acid, C12). FIG. 4f shows the production of γ-tridecalactone (GC13) from tridecylic acid (tridecanoic acid, C13). FIG. 4g shows the production of γ-tetradecalactone (GC14) from myristic acid (tetradecanoic acid, C14).

### DETAILED DESCRIPTION

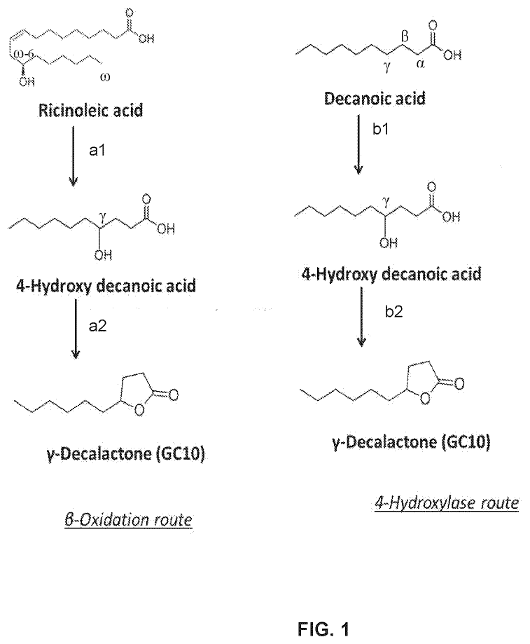

Referring to FIG. 1, biosynthetic production of gamma lactones from fatty acids can be performed via at least two different routes. According to the β-oxidation route, a medium-chain (C6-C12) or long-chain fatty acid (C13 to C21) having a hydroxyl group close to the omega carbon (e.g., ω-1, ω-2, ω-3, ω-4, ω-5, ω-6 etc.) can be incubated with a yeast to cause multiple cycle of β-oxidation (step a1), with each β-oxidation cycle removing a C2 residue from the hydrocarbon backbone fragment between the hydroxylated carbon atom and the carbonyl carbon atom. Depending whether there is an odd or even number of carbon atoms within such hydrocarbon backbone fragment, a 4-hydroxy (gamma-hydroxy) or a 5-hydroxy (delta-hydroxy) fatty acid may result, and upon lactonization under acidic conditions (step a2), either a gamma lactone or a delta lactone is produced.

By comparison, the present method makes use of cytochrome P450 proteins that have been specifically identified with 4-hydroxylase activity. Carboxylic acids or derivatives thereof can be bioconverted by such cytochrome P450 proteins into 4-hydroxy carboxylic acids (step b1), which upon acidification (step b2), produce the corresponding gamma lactone. The present methods, therefore, provide an economical and reliable approach for producing gamma lactones that is suitable for commercial scale-up production.

### Methods of Making Lactones

Methods described herein, in some embodiments, provide for the production of C4-C20 gamma lactones from a C4-C20 carboxylic acid substrate. In some embodiments, methods of making such gamma lactones comprise (a) incubating a cellular system expressing a heterologous cytochrome P450 (CYP450) protein with a C4-C20 carboxylic acid substrate to provide a 4-hydroxy C4-C20 carboxylic acid, and (b) contacting or otherwise subjecting the 4-hydroxy carboxylic acid to acidic conditions to produce the C4-C20 gamma lactone.

In some embodiments, methods of making the lactones comprise incubating cell pellets harvested from the cellular system expressing the heterologous CYP450 protein with the carboxylic acid substrate. In some embodiments, methods of making the lactones comprise incubating resuspended cell pellets harvested from the cellular system with the carboxylic acid substrate.

Methods of making gamma lactones described herein comprise incubating a cellular system expressing a heterologous CYP450 protein in a medium comprising the carboxylic acid substrate at any ratio. In some embodiments, methods of making gamma lactones comprise incubating a cellular system comprising between 1 gram of cells per liter to 200 grams of cells per liter (1 g/L to 200 g/L) of the medium. The medium can include between 1 gram of carboxylic acid substrate per liter to 20 grams of fatty acids per liter (1 g/L to 20 g/L). In some embodiments, methods of making lactones comprise incubating a cellular system comprising 100 grams of cells per liter (100 g/L) in a medium that includes 1 gram of carboxylic acid substrate per liter (1 g/L).

In some embodiments, methods of making lactones comprise incubating a cellular system with a medium comprising the carboxylic acid substrate for a suitable period sufficient for the formation of 4-hydroxy carboxylic acid. In some embodiments, methods of making lactones comprise contacting the 4-hydroxy carboxylic acids with a cell culture for a suitable period sufficient for formation of gamma lactones in the cell culture. In some embodiments, methods of making lactones comprise isolating the 4-hydroxy carboxylic acids prior to contact with the cell culture. In some embodiments, methods of making lactones comprise incubating the 4-hydroxy carboxylic acids in a medium with the cell culture.

Methods of making lactones described herein encompass incubating (e.g., incubating a cellular system expressing a heterologous CYP450 protein with a medium comprising the appropriate carboxylic acid substrate) for an amount of time. In some embodiments, methods of making lactones comprise incubating for a time between 0.5 hour to 96 hours.

Methods of making lactones described herein encompass incubating (e.g., incubating a cellular system expressing a heterologous CYP450 protein with a medium comprising the appropriate carboxylic acid substrate) at a temperature. In some embodiments, methods of making lactones comprise incubating at a temperature between 16° C. to 40° C.

### Cytochrome P450

Cytochrome P450 (CYP450) proteins, generally, are enzymes that catalyze oxidation of substrates. CYP450 proteins are members of the hemeprotein superfamily, and contain heme as a cofactor. CYP450 enzymes have been identified in many organisms including, but not limited to, animals, plants, fungi, bacteria, archaea, and viruses. However, to the inventors' knowledge, previous to the current invention, CYP450 enzymes having specific carboxylic acid 4-hydroxylase activity have not been reported.

After performing rigorous high-throughput screening of large libraries of candidate genes for fungal cytochrome P450 enzymes with carboxylic acid 4-hydroxylase activity, the inventors have identified certain CYP450 genes that show high carboxylic acid 4-hydroxylase activity.

A CYP450 protein suitable for use in the present methods can be a Mucor ambiguous CYP450 protein or a functional variant thereof that has carboxylic acid 4-hydroxylase activity. Specifically, the CYP450 protein can comprise an amino acid sequence having at least 80%, 85%, 90%, 95%, 96%, 97%, 98%, or 99% identity to SEQ ID NO: 1. In certain embodiments, the CYP450 protein can comprise the amino acid sequence of SEQ ID NO: 1. In other embodiments, the CYP450 protein can consist of the amino acid sequence of SEQ ID NO: 1. Accordingly, the cellular system used to express such heterologous CYP450 protein can comprise host cells having a nucleotide sequence having at least 80%, 85%, 90%, 95%, 97%, 98%, or 99% identity to SEQ ID NO: 2.

In some other embodiments, the CYP450 protein can be a Basidiobolus meristosporus CYP450 protein or a functional variant thereof that has carboxylic acid 4-hydroxylase activity. For example, the CYP450 protein can comprise an amino acid sequence having at least 80%, 85%, 90%, 95%, 96%, 97%, 98%, or 99% identity to SEQ ID NO: 3. In certain embodiments, the CYP450 protein can comprise the amino acid sequence of SEQ ID NO: 3. In other embodiments, the CYP450 protein can consist of the amino acid sequence of SEQ ID NO: 3. Accordingly, the cellular system used to express such heterologous CYP450 protein can comprise host cells having a nucleotide sequence having at least 80%, 85%, 90%, 95%, 97%, 98%, or 99% identity to SEQ ID NO: 4. In yet other embodiments, the CYP450 protein can be a Umbelopsis isabelline CYP450 protein or a functional variant thereof that has carboxylic acid 4-hydroxylase activity. For example, the CYP450 protein can comprise an amino acid sequence having at least 80%, 85%, 90%, 95%, 96%, 97%, 98%, or 99% identity to SEQ ID NO: 5. In certain embodiments, the CYP450 protein can comprise the amino acid sequence of SEQ ID NO: 5. In other embodiments, the CYP450 protein can consist of the amino acid sequence of SEQ ID NO: 5. Accordingly, the cellular system used to express such heterologous CYP450 protein can comprise host cells having a nucleotide sequence having at least 80%, 85%, 90%, 95%, 97%, 98%, or 99% identity to SEQ ID NO: 6.

### Carboxylic Acid Substrates

According to methods described herein, a C4-C20 carboxylic acid substrate can be converted to 4-hydroxy carboxylic acids. The carboxylic acid substrate can be a carboxylic acid and/or a derivative thereof. For example, the C4-C20 carboxylic acid substrate can include a C4-C20 carboxylic acid; a salt of a C4-C20 carboxylic acid; an ester of a C4-C20 carboxylic acid; a mono-, di- or triglyceride of a C4-C20 carboxylic acid; or a combination thereof. As used herein, C4-C20 denotes an organic compound having 4-20 carbon atoms. The range expressed by C4-C20 is meant to convey any ranges therewithin, including but not limited to C5-C20, C6-C20, C7-C20, C8-C20, C9-C20, C10-C20, C6-C14, C7-C14, C8-C14, C9-C14, C10-C14 etc. Accordingly, the carboxylic acid substrate described herein can include a carboxylic acid or a derivative thereof having 4, 5, 6, 7, 8, 9, 10, 11, 12, 13, 14, 15, 16, 17, 18, 19, or 20 carbon atoms. In certain embodiments, the C4-C20 carboxylic acid substrate can be a C4-C20 carboxylic acid or a salt thereof. Without limitation, the substrate acid can be in the form of its sodium salt, potassium salt, calcium salt, magnesium salt, ammonium salt, and the like.

The C4-C20 carboxylic acid can be a straight-chain C4-C20 carboxylic acid, a branched C4-C20 carboxylic acid, a saturated C4-C20 carboxylic acid, or an unsaturated C4-C20 carboxylic acid. The C4-C20 carboxylic acid can be optionally substituted with one or more functional groups selected from a hydroxyl group and an amino group, provided that such optional substitution is not at the carbon atoms that are positioned alpha, beta, or gamma to the carbonyl carbon of the carboxylic acid (see FIG. 1). In certain embodiments, the C4-C20 carboxylic acid can be a straight-chain fully saturated carboxylic acid.

Examples of C4-C20 carboxylic acids include, but are not limited to, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid, heptatriacontanoic acid, octatriacontanoic acid, nonatriacontanoic acid, and tetracontanoic acid. Table 1 provides common names, structural formulas, and lipid numbers of exemplary fatty acids.

TABLE 1
Examples of fatty acids.

| | | | Lipid |
|---|---|---|---|
| Common Name | Systematic Name | Structural Formula | Numbers |
| Valeric acid | Pentanoic acid | CH₃(CH₂)₃COOH | C5:0 |
| Caproic acid | Hexanoic acid | CH₃(CH₂)₄COOH | C6:0 |
| Enanthic acid | Heptanoic acid | CH₃(CH₂)₅COOH | C7:0 |
| Caprylic acid | Octanoic acid | CH₃(CH₂)₆COOH | C8:0 |
| Pelargonic acid | Nonanoic acid | CH₃(CH₂)₇COOH | C9:0 |
| Capric acid | Decanoic acid | CH₃(CH₂)₈COOH | C10:0 |
| Undecylic acid | Undecanoic acid | CH₃(CH₂)₉COOH | C11:0 |
| Lauric acid | Dodecanoic acid | CH₃(CH₂)₁₀COOH | C12:0 |
| Tridecylic acid | Tridecanoic acid | CH₃(CH₂)₁₁COOH | C13:0 |
| Myristic acid | Tetradecanoic acid | CH₃(CH₂)₁₂COOH | C14:0 |
| Pentadecylic acid | Pentadecanoic acid | CH₃(CH₂)₁₃COOH | C15:0 |
| Palmitic acid | Hexadecanoic acid | CH₃(CH₂)₁₄COOH | C16:0 |
| Margaric acid | Heptadecanoic acid | CH₃(CH₂)₁₅COOH | C17:0 |
| Stearic acid | Octadecanoic acid | CH₃(CH₂)₁₆COOH | C18:0 |
| Nonadecylic acid | Nonadecanoic acid | CH₃(CH₂)₁₇COOH | C19:0 |
| Arachidic acid | Eicosanoic acid | CH₃(CH₂)₁₈COOH | C20:0 |
| Heneicosylic acid | Heneicosanoic acid | CH₃(CH₂)₁₉COOH | C21:0 |
| Behenic acid | Docosanoic acid | CH₃(CH₂)₂₀COOH | C22:0 |
| Tricosylic acid | Tricosanoic acid | CH₃(CH₂)₂₁COOH | C23:0 |
| Lignoceric acid | Tetracosanoic acid | CH₃(CH₂)₂₂COOH | C24:0 |
| Pentacosylic acid | Pentacosanoic acid | CH₃(CH₂)₂₃COOH | C25:0 |
| Cerotic acid | Hexacosanoic acid | CH₃(CH₂)₂₄COOH | C26:0 |
| Heptacosylic acid | Heptacosanoic acid | CH₃(CH₂)₂₅COOH | C27:0 |
| Montanic acid | Octacosanoic acid | CH₃(CH₂)₂₆COOH | C28:0 |
| Nonacosylic acid | Nonacosanoic acid | CH₃(CH₂)₂₇COOH | C29:0 |
| Melissic acid | Triacontanoic acid | CH₃(CH₂)₂₈COOH | C30:0 |
| Henatriacontylic acid | Henatriacontanoic acid | CH₃(CH₂)₂₉COOH | C31:0 |
| Lacceroic acid | Dotriacontanoic acid | CH₃(CH₂)₃₀COOH | C32:0 |
| Psyllic acid | Tritriacontanoic acid | CH₃(CH₂)₃₁COOH | C33:0 |
| Geddic acid | Tetratriacontanoic acid | CH₃(CH₂)₃₂COOH | C34:0 |
| Ceroplastic acid | Pentatriacontanoic acid | CH₃(CH₂)₃₃COOH | C35:0 |
| Hexatriacontylic acid | Hexatriacontanoic acid | CH₃(CH₂)₃₄COOH | C36:0 |
| Heptatriacontanoic acid | Heptatriacontanoic acid | CH₃(CH₂)₃₅COOH | C37:0 |
| Octatriacontanoic acid | Octatriacontanoic acid | CH₃(CH₂)₃₆COOH | C38:0 |
| Nonatriacontanoic acid | Nonatriacontanoic acid | CH₃(CH₂)₃₇COOH | C39:0 |
| Tetracontanoic acid | Tetracontanoic acid | CH₃(CH₂)₃₈COOH | C40:0 |

Examples of unsaturated C4-C20 carboxylic acids include hexenoic acid, 1-heptenoic acid, 2-heptenoic acid, 1-octenoic acid, 2-octenoic acid, 3-octenoic acid, 1-nonenoic acid, 2-nonenoic acid, 3-nonenoic acid, 1-decenoic acid, 2-decenoic acid, 3-decenoic acid, 1-undecenoic acid, 2-undecenoic acid, 3-undecenoic acid, 1-dodecenoic acid, 2-dodecenoic acid, 3-dodecenoic acid, 1-tridecenoic acid, 2-tridecenoic acid, 3-tridecenoic acid, 1-tetradecenoic acid, 2-tetradecenoic acid, and 3-tetradecenoic acid.

In some embodiments, the medium comprising the carboxylic acid substrate comprises a buffer. Examples of buffers include, without limitation, phosphate buffer, Tris buffer, MOPS buffer, HEPES buffer, citrate buffer, acetate buffer, malate buffer, MES buffer, histidine buffer, PIPES buffer, bis-tris buffer, and ethanolamine buffer.

In some embodiments, the medium comprising the carboxylic acid substrate comprises a surfactant to help dispersion of the substrate. Non-limiting examples of surfactants include polysorbate 20 (TWEEN® 20), polyoxyethylenesorbitan monopalmitate (TWEEN® 40), 4-(1,1,3,3-Tetramethylbutyl)phenyl-polyethylene glycol (TRITON™ X-100), sodium dodecyl sulfate (SDS), ethyl trimethylammonium bromide (ETMAB), lauryl trimethyl ammonium bromide (LTAB), and lauryl trimethylammonium chloride (LTAC).

A medium comprising the carboxylic acid substrate may comprise any concentration of the carboxylic acid substrate. In some embodiments, the medium comprising the carboxylic acid substrate can include 0.5 g/L to 100 g/L of the carboxylic acid substrate, e.g., 1 g/L to 10 g/L of the carboxylic acid substrate. In some embodiments, the medium comprising the carboxylic acid substrate can include 0.5 g/L, 1 g/L, 2 g/L, 3 g/L, 4 g/L, 5 g/L, 6 g/L, 7 g/L, 8 g/L, 9 g/L, 10 g/L, 20 g/L, 30 g/L, 40 g/L, 50 g/L, 60 g/L, 70 g/L, 80 g/L, 90 g/L or 100 g/L of the carboxylic acid substrate.

### Lactones

According to methods described herein, the carboxylic acid substrate is converted to a 4-hydroxy carboxylic acid in the presence of the CYP450 protein described herein. Upon acidification, the 4-hydroxy carboxylic acid can be converted to the corresponding gamma lactone. Acidification can be carried out with an acid such as hydrochloric acid, acetic acid, and the like. According to the present disclosure, the methods provided herein can convert 4-hydroxy carboxylic acids into various γ-lactones, including without limitation, γ-hexalactone, γ-hexenolactone, γ-heptalactone, γ-heptenolactone, γ-octalactone, γ-octenolactone, γ-nonalactone, γ-nonenolactone, γ-decalactone, γ-decacenolactone, γ-undecalactone, γ-undecenolactone, γ-dodecalactone, γ-dodecenolactone, γ-tridecalactone, γ-tridecenolactone, γ-tetradecalactone, and γ-tetradecenolactone.

The gamma lactones produced according to methods described herein can be isolated or purified from the cell culture using any method known in the art. For example, the gamma lactones can be isolated or purified from the cell culture by solvent extraction, distillation, chromatographic separation, high pressure liquid chromatography and the like. The isolated or purified lactones can be incorporated into a product, e.g., a food, a beverage, a perfume or a cosmetic product.

### Cellular Systems

As referred herein, a cellular system according to the present methods can include any cell or cells that provide for expression of the CYP450 proteins described herein. Such cellular system can include, but are not limited to, bacterial cells, yeast cells, plant cells, and animal cells. In some embodiments, the cellular system comprises bacterial cells, yeast cells, or a combination thereof. In some embodiments, the cellular system comprises prokaryotic cells, eukaryotic cells, and combinations thereof. In some embodiments, the cellular system comprises in vitro expression of proteins based on cellular components, such as ribosomes.

Bacterial cells of the present disclosure include, without limitation, Escherichia spp., Streptomyces spp., Zymomonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., Pantoea spp., and Vibrio natriegens.

Yeast cells of the present disclosure include, without limitation, engineered Saccharomyces spp., Schizosaccharomyces, Hansenula, Candida, Kluyveromyces, Yarrowia, Candida boidinii, and Pichia. According to the current disclosure, a yeast as claimed herein are eukaryotic, single-celled microorganisms classified as members of the fungus kingdom. Yeasts are unicellular organisms which evolved from multicellular ancestors but with some species useful for the current disclosure being those that have the ability to develop multicellular characteristics by forming strings of connected budding cells known as pseudo hyphae or false hyphae.

In some embodiments, cell pellets are harvested from the cellular system expressing CYP450. In some embodiments, the cell pellets may be resuspended at various concentrations. In some embodiments, the cell pellets are resuspended at a concentration of 1 g/L to 250 g/L. In some embodiments, the cell pellets harvested from the cellular system are resuspended at a concentration of 1 g/L, 10 g/L, 25 g/L, 50 g/L, 75 g/L, 100 g/L, 125 g/L, 150 g/L, 175 g/L, 200 g/L, 225 g/L, or 250 g/L.

### Cell Culture

A cell culture refers to any cell or cells that are in a culture. Culturing or incubating is the process in which cells are grown under controlled conditions, typically outside of their natural environment. For example, cells, such as yeast cells, may be grown as a cell suspension in liquid nutrient broth. A cell culture includes, but is not limited to, a bacterial cell culture, a yeast cell culture, a plant cell culture, and an animal cell culture. In some embodiments, the cell culture comprises bacterial cells, yeast cells, or a combination thereof.

A bacterial cell culture of the present disclosure comprises bacterial cells including, but not limited to, Escherichia spp., Streptomyces spp., Zymomonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., Pantoea spp, and Vibrio natriegens.

A yeast cell culture of the present disclosure comprises yeast cells including, but not limited to Saccharomyces spp., Schizosaccharomyces, Hansenula, Candida, Kluyveromyces, Yarrowia, Candida boidinii, and Pichia.

In some embodiments, a cell culture as described herein can be an aqueous medium including one or more nutrient substances as known in the art. Such liquid medium can include one or more carbon sources, nitrogen sources, inorganic salts, and/or growth factors. Suitable carbon sources can include glucose, fructose, xylose, sucrose, maltose, lactose, mannitol, sorbitol, glycerol, and corn syrup. Examples of suitable nitrogen sources can include organic and inorganic nitrogen-containing substances such as peptone, corn steep liquor, mean extract, yeast extract, casein, urea, amino acids, ammonium salts, nitrates and mixtures thereof. Examples of inorganic salts can include phosphates, sulfates, magnesium, sodium, calcium, and potassium salts. The liquid medium also can include one or more vitamins and/or minerals.

In some embodiments, cells are cultured at a temperature of 16° C. to 40° C. For example, cells may be cultured at a temperature of 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. or 40° C.

In some embodiments, cells are cultured at a pH range from about 3 to about 9, preferably in the range of from about 4 to about 8. The pH can be regulated by the addition of an inorganic or organic acid or base such as hydrochloric acid, acetic acid, sodium hydroxide, calcium carbonate, ammonia, or by the addition of a buffer such as phosphate, phthalate or Tris®.

In some embodiments, cells are cultured for a period of time of 0.5 hours to 96 hours, or more. For example, cells may be cultured for a period of time of 12, 18, 24, 30, 36, 42, 48, 54, 60, 66, or 72 hours. Typically, cells, such as bacterial cells, are cultured for a period of time of 12 to 24 hours. In some embodiments, cells are cultured for 12 to 24 hours at a temperature of 37° C. In some embodiments, cells are cultured for 12 to 24 hours at a temperature of 16° C.

In some embodiments, cells are cultured to a density of 1×10 8 (OD₆₀₀<1) to 2×10¹¹ (OD˜200) viable cells/ml cell culture medium. In some embodiments, cells are cultured to a density of 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4 x 1°, 5×10¹⁰, 6×10¹⁰, 7×10¹⁰, 8×10¹⁰, 9×10¹⁰, 1×10¹¹, or 2×10¹¹ viable cells/ml. (Conversion factor: OD 1=8×10⁸ cells/ml).

### Synthetic Biology

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and Ausubel, F. M. et al., IN CURRENT PROTOCOLS IN MOLECULAR BIOLOGY, published by GREENE PUBLISHING AND WILEY-INTERSCIENCE, 1987; the entirety of each of which is hereby incorporated herein by reference.

### Bacterial Production Systems

Expression of proteins in prokaryotes is most often carried out in a bacterial host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

In an embodiment, the expression vector includes those genetic elements for expression of the recombinant polypeptide in bacterial cells. The elements for transcription and translation in the bacterial cell can include a promoter, a coding region for the protein complex, and a transcriptional terminator.

Persons of ordinary skill in the art will be aware of the molecular biology techniques available for the preparation of expression vectors. The polynucleotide used for incorporation into the expression vector of the subject technology, as described herein, can be prepared by routine techniques such as polymerase chain reaction (PCR).

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities and fill-in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared by the use of PCR using appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into plant or microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector,

In some embodiments, the transformed cell can be a bacterial cell, a yeast cell, an algal cell, a fungal cell, a plant cell, an insect cell or an animal cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell expression systems and expression vectors containing regulatory sequences that direct high-level expression of foreign proteins are well known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYCI, HIS3, GALI, GALIO, ADHI, PGK, PH05, GAPDH, ADCI, TRPI, URA3, LEU2, ENO, TPI (useful for expression in Saccharomyces); AOXI (useful for expression in Pichia); and lac, trp, JPL, IPR, T7, tac, and trc (useful for expression in Escherichia coli).

Termination control regions may also be derived from various genes native to the microbial hosts. A termination site optionally may be included for the microbial hosts described herein.

In plant cells, the expression vectors of the subject technology can include a coding region operably linked to promoters capable of directing expression of the recombinant polypeptide of the subject technology in the desired tissues at the desired stage of development. For reasons of convenience, the polynucleotides to be expressed may comprise promoter sequences and translation leader sequences derived from the same polynucleotide. 3′ non-coding sequences encoding transcription termination signals should also be present. The expression vectors may also comprise one or more introns in order to facilitate polynucleotide expression.

For plant host cells, any combination of any promoter and any terminator capable of inducing expression of a coding region may be used in the vector sequences of the subject technology. Some suitable examples of promoters and terminators include those from nopaline synthase (nos), octopine synthase (ocs) and cauliflower mosaic virus (CaMV) genes. One type of efficient plant promoter that may be used is a high-level plant promoter. Such promoters, in operable linkage with an expression vector of the subject technology should be capable of promoting the expression of the vector. High level plant promoters that may be used in the subject technology include the promoter of the small subunit (s) of the ribulose-1,5-bisphosphate carboxylase for example from soybean (Berry-Lowe et al., J. MOLECULAR AND APP. GEN., 1:483-98 (1982), the entirety of which is hereby incorporated herein to the extent it is consistent herewith), and the promoter of the chlorophyll binding protein. These two promoters are known to be light-induced in plant cells (see, for example, GENETIC ENGINEERING OF PLANTS, AN AGRICULTURAL PERSPECTIVE, A. Cashmore, Plenum, N.Y. (1983), pages 29-38; Coruzzi, G. et al., THE JOURNAL OF BIOLOGICAL CHEMISTRY, 258: 1399 (1983), and Dunsmuir, P. et al., JOURNAL OF MOLECULAR AND APPLIED GENETICS, 2:285 (1983), each of which is hereby incorporated herein by reference to the extent they are consistent herewith).

### Analysis of Sequence Similarity Using Identity Scoring

As used herein, “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this disclosure “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch (Needleman and Wunsch, JOURNAL OF MOLECULAR BIOLOGY 48:443-53, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-10 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the disclosure is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein. Polynucleotide molecules that have the activity of the Cytochrome P450 genes of the current disclosure are capable of directing the production of a variety of γ- and delta-lactones, Such polynucleotide molecules can have a substantial percent sequence identity to the polynucleotide sequences provided herein and are encompassed within the scope of this disclosure.

### Identity and Similarity

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

### Explanation of Terms Used Herein:

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

“Coding sequence” is to be given its ordinary and customary meaning to a person of ordinary skill in the art, and is used without limitation to refer to a DNA sequence that encodes for a specific amino acid sequence.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing the desired product.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

“Percent (%) amino acid sequence identity” with respect to the variant polypeptide sequences of the subject technology refers to the percentage of amino acid residues in a candidate sequence that are identical with the amino acid residues of a reference polypeptide, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, the % amino acid sequence identity may be determined using the sequence comparison program NCBI-BLAST2. The NCBI-BLAST2 sequence comparison program may be downloaded from ncbi.nlm.nih.gov. NCBI BLAST2 uses several search parameters, wherein all of those search parameters are set to default values including, for example, unmask yes, strand=all, expected occurrences 10, minimum low complexity length=15/5, multi-pass e-value=0.01, constant for multi-pass=25, dropoff for final gapped alignment=25 and scoring matrix=BLOSUM62. In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

In this sense, techniques for determining amino acid sequence “similarity” are well known in the art. In general, “similarity” refers to the exact amino acid to amino acid comparison of two or more polypeptides at the appropriate place, where amino acids are identical or possess similar chemical and/or physical properties such as charge or hydrophobicity. A so-termed “percent similarity” may then be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded therein, and comparing this to a second amino acid sequence. In general, “identity” refers to an exact nucleotide to nucleotide or amino acid to amino acid correspondence of two polynucleotides or polypeptide sequences, respectively. Two or more polynucleotide sequences can be compared by determining their “percent identity”, as can two or more amino acid sequences. The programs available in the Wisconsin Sequence Analysis Package, Version 8 (available from Genetics Computer Group, Madison, Wis.), for example, the GAP program, are capable of calculating both the identity between two polynucleotides and the identity and similarity between two polypeptide sequences, respectively. Other programs for calculating identity or similarity between sequences are known by those skilled in the art.

An amino acid position “corresponding to” a reference position refers to a position that aligns with a reference sequence, as identified by aligning the amino acid sequences. Such alignments can be done by hand or by using well-known sequence alignment programs such as ClustalW2, Blast 2, etc.

Unless specified otherwise, the percent identity of two polypeptide or polynucleotide sequences refers to the percentage of identical amino acid residues or nucleotides across the entire length of the shorter of the two sequences.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art, and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of reasonable skill in the field, and is used without limitation to refer to the transfer of a polynucleotide into a target cell for further expression by that cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “recombinant” or “transformed” organisms.

The terms “transformed,” “transgenic,” and “recombinant,” when used herein in connection with host cells, are to be given their respective ordinary and customary meanings to a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### EXAMPLES

### Example 1: Identifying Candidate Genes for Fungal Cytochrome P450 Enzymes with Fatty Acid 4-Hydroxylase Activity

Cytochrome P450 (CYP) enzymes can catalyze the oxidation of a wide variety of organic substrates (including fatty acids) to generate various hydroxylated compounds (including hydroxy fatty acids). However, the position of the carbon atom at which the hydroxylation takes place along the hydrocarbon backbone can be critical. For example, among various P450s, bacterial and fungal P450 BM3 enzymes have been reported to catalyze the formation of ω-1 to ω-3 hydroxy fatty acids. See e.g., Miura, Y. and Fulco, A. J., “ω-1, ω-2 and ω-3 Hydroxylation of Long-Chain Fatty Acids, Amides and Alcohols by a Soluble Enzyme System from Bacillus megatyerium,” Biochimica et Biophysica Acta—Lipids and Lipid Metabolism, 388(3): 305-317 (1975); and Kitazume, T. et al., “Fusarium oxysporum Fatty-acid Subterminal Hydroxylase (CYP505) Is a Membrane-bound Eukaryotic Counterpart of Bacillus megaterium Cytochrome P450BM3,” J. Bio. Chem., 275: 39734-39740 (2000).

Referring to FIG. 1, an efficient synthetic pathway from various fatty acids to gamma-lactones involve the hydroxylation of the carbon atom positioned gamma to the carbonyl carbon of the fatty acid substrate, thereby generating a 4-hydroxy fatty acid (4-hydroxylase route). Subsequently, the 4-hydroxy fatty acid can be converted into a gamma-lactone under acidic conditions.

It has been reported that some filamentous fungi such as Mucor circinelloides, Aspergillus oryzae, and Mortierella isabellina can make various gamma- or delta-lactones from the corresponding carboxylic acids. See U.S. Pat. Nos. 5,032,513 and 7,863,023. Nevertheless, the molecular mechanisms for their bioconversion activities are largely unknown. In addition, the complex morphology of such filamentous fungi makes the scale-up of these processes a big challenge.

To the inventors' knowledge, previous to the current invention, no specific fatty acid 4-hydroxylase genes or enzymes have been identified. After performing rigorous high-throughput screening of large libraries of candidate genes for fungal cytochrome P450 (CYP, P450, or CYP450) enzymes with fatty acid 4-hydroxylase activity, the inventors identified the following candidate genes: a cytochrome P450 monooxygenase gene (GenBank: GAN03094.1) from Mucor ambiguus, the amino acid sequence of said Mucor ambiguus CYP is provided as SEQ ID NO: 1; a cytochrome P450 monooxygenase gene (GenBank: ORX85448.1) from Basidiobolus meristosporus, the amino acid sequence of said Basidiobolus meristosporus CYP is provided as SEQ ID NO: 3; and a cytochrome P450 monooxygenase gene (GenBank: ORX85448.1) from Umbelopsis isabellina, the amino acid sequence of said Umbelopsis isabellina CYP is provided as SEQ ID NO: 5.

### Example 2: Generation of High Yield Strains and Overexpression of Cytochrome P450 for Producing Gamma-Lactones

To facilitate protein expression in E. coli, each of the candidate genes was codon-optimized for E. coli genome and synthesized by Gene Universal Inc. (Newark, DE).

The codon-optimized Mucor ambiguus P450 gene was cloned into a pET17b vector (AMP+, Novagen) through HindIII and XhoI sites. The construct was transformed into BL21(DE3) cells for expression. In a typical experiment, an overnight culture was used to inoculate liquid LB medium (2%) containing 100 mg/L of carbenicillin. The culture was first grown at 37° C. to an OD600 of 0.6 and cooled down to 25° C. Then 1 mM of IPTG was added to induce protein expression. After 3 hours of incubation at 25° C., the fatty acid substrate was added at 1 g/L for γ-lactone production.

Each of the codon-optimized Basidiobolus meristosporus P450 and Umbelopsis isabellina P450 genes was cloned into pET-32a-(+) vector (AMP+, Novagen) through HindIII and XhoI sites. The construct was transformed into BL21(DE3) cells for expression. In a typical experiment, an overnight culture was used to inoculate liquid LB medium (2%) containing 100 mg/L of carbenicillin. The culture was first grown at 37° C. to an OD600 of 0.8 and cooled down to 16° C. Then 1 mM of IPTG was added to induce protein expression. After 5 h of incubation at 16° C., the temperature was raised to 30° C. and the fatty acid substrate was added at 1 g/L for γ-lactone production.

### Example 3: Analysis of Gamma-Lactone Production

To analyze γ-lactone production, 0.5 ml of the E. coli culture described in Example 2 was taken and acidified with 10 μl of 2N HCl, then extracted with 0.5 ml of ethyl acetate with shaking at room temperature for 60 min. After centrifugation at 14,000 rpm for 15 min, the ethyl acetate phase was used for GC/MS or GC/FID analysis.

GC/MS analysis was conducted on Shimadzu GC-2010 system coupled with GCMS-QP2010S detector. The analytical column is SHRXI-5MS (thickness 0.25 μm; length 30 m; diameter 0.25 mm) and the injection temperature is 265° C. under split mode. The temperature gradient is 0-3 min 80° C.; 3-8.7 min 120° C. to 263° C., a gradient of 25; 8.7-10.7 min, 263° C.

GC/FID analysis was conducted on Shimadzu GC-2014 system. The analytical column is Restek RXi-5 ms (thickness 0.25 μm; length 30 m; diameter 0.25 mm) and the injection temperature is 240° C. under split mode. The temperature gradient is 0-3 min, 100° C.; 3-9 min 100° C.-280° C., a gradient of 30; 9-12 min, 280° C.

### Example 4: Gamma-Lactone Production Using High Yield Strains Overexpressing Mucor ambiguus P450

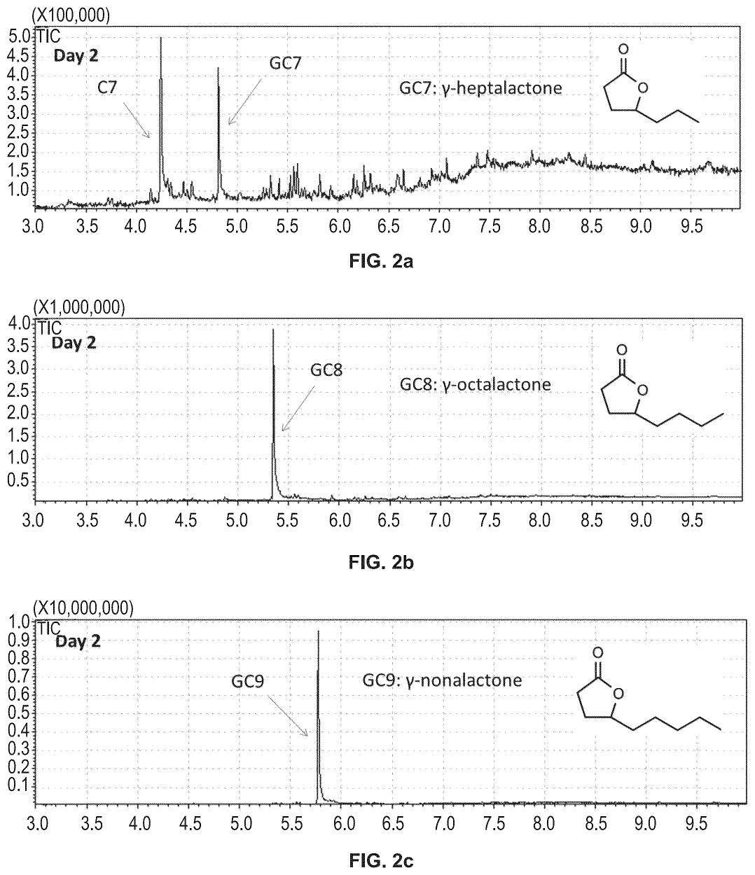

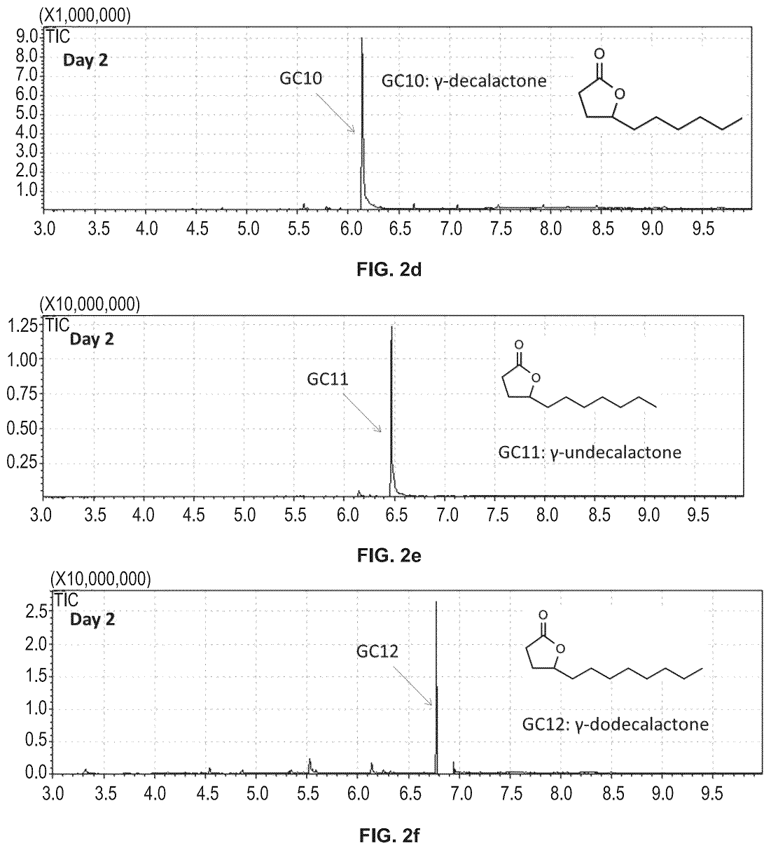

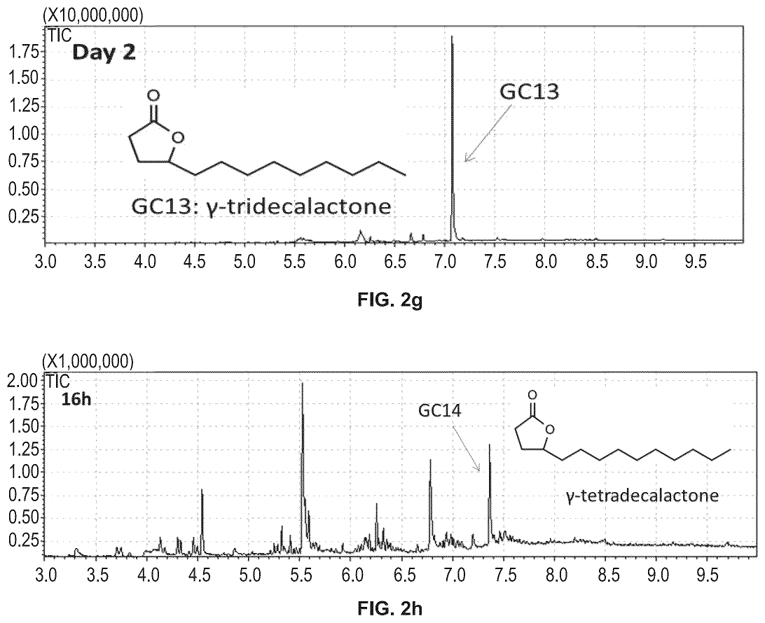

The procedures described in Example 2 were performed using an E. coli culture overexpressing Mucor ambiguus P450. Specifically, samples of the E. coli culture were taken and acidified 2 days or 16 h after adding the fatty acid substrate. FIGS. 2a-2h shows the production of various gamma-lactones from the corresponding carboxylic acid: (a) γ-heptalactone (GC7) from enanthic acid (heptanoic acid, C7), (b) γ-octalactone (GC8) from caprylic acid (octanoic acid, C8), (c) γ-nonalactone (GC9) from pelargonic acid (nonanoic acid, C9), (d) γ-decalactone (GC10) from capric acid (decanoic acid, C10), (e) γ-undecalactone (GC11) from undecylic acid (undecanoic acid, C11), (f) γ-dodecalactone (GC12) from lauric acid (dodecanoic acid, C12), (g) γ-tridecalactone (GC13) from tridecylic acid (tridecanoic acid, C13), and (h) γ-tetradecalactone (GC14) from myristic acid (tetradecanoic acid, C14). As shown in FIG. 2a to FIG. 2h, GC7-GC14 γ-lactones were effectively produced from the corresponding C7-C14 carboxylic acids in a heterologous system that overexpressed Mucor ambiguus cytochrome P450 (SEQ ID NO: 1). Notably, the GC7 to GC12 peaks match the corresponding standards from Sigma-Aldrich (St Louis, MO) in terms of both retention times and mass spectra. The standards for γ-tridecalactone (GC13) and γ-tetradecalactone (GC14) are not commercially available. Nevertheless, it was noted that the putative GC13 and GC14 peaks have retention times longer than that of GC12.

### Example 5: Gamma-Lactone Production Using High Yield Strains Overexpressing Basidiobolus meristosporus P450

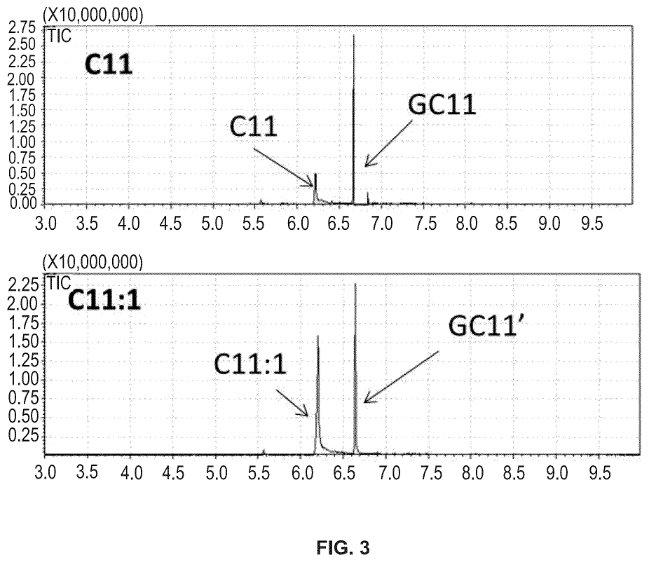

The procedures described in Example 2 were performed using an E. coli culture overexpressing Basidiobolus meristosporus P450. Specifically, samples of the E. coli culture were taken and acidified 17 hours after adding the fatty acid substrate. FIG. 3 shows the production of various gamma-lactones from the corresponding carboxylic acid: (a) γ-undecalactone (GC11) from undecylic acid (undecanoic acid, C11), and (b) γ-undecenolactone (GC11′) from undecylenic acid (undecenoic acid, C11:1).

As shown in FIG. 3, GC11 and GC11′ γ-lactones were effectively produced from the corresponding C11 and C11:1 carboxylic acids in a heterologous system that overexpressed Basidiobolus meristosporus cytochrome P450 (SEQ ID NO: 3). Notably, the C11, GC11 and C11:1 peaks match the corresponding standards from Sigma-Aldrich (St Louis, MO) in terms of both retention times and mass spectra. The standard for GC11′ is not commercially available. Nevertheless, it was noted that the putative GC11′ peak shows a retention time similar to that of GC11.

Under the reaction conditions described, about 69 mg/L of GC11 and about 18 mg/L of GC11′ were produced 17 hours after adding 1 g/L of C11 or C11:1 to the E. coli growing cell cultures, respectively.

In addition to growing cells, resting cells were also investigated for their potential use in the bioconversion of fatty acids into gamma-lactones. In this experiment, an overnight culture was used to inoculate liquid SOC medium (3%) containing 100 mg/L of carbenicillin. The culture was first grown at 37° C. to an OD600 of 0.8 and cooled down to 16° C. Then, 1 mM of IPTG was added to induce protein expression. After 16h of incubation at 16° C., the culture was harvested and cell pellets were collected by centrifugation and stored at −80° C. until use. In a typical bioconversion experiment, the frozen cells were re-suspended in 100 mM K-Pi buffer, pH7 at a cell concentration of 100 g/L fresh weight. Then, 1 g/L of C11 or C11:1 was added together with 0.1% of Tween 40 and 10 mM of NADPH. Subsequently, the mixture was incubated at 30° C. with shaking at 250 rpm. Samples were taken 3h later. The titers for GC11 and GC11′ were 419 mg/L and 185 mg/L, respectively.

### Example 6: Gamma-Lactone Production Using High Yield Strains Overexpressing Umbelopsis isabellina P450

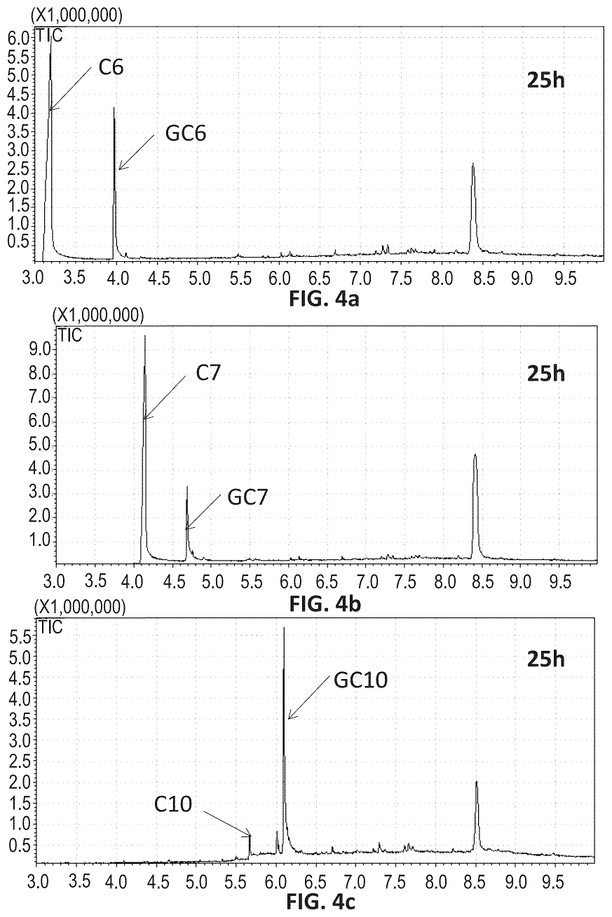

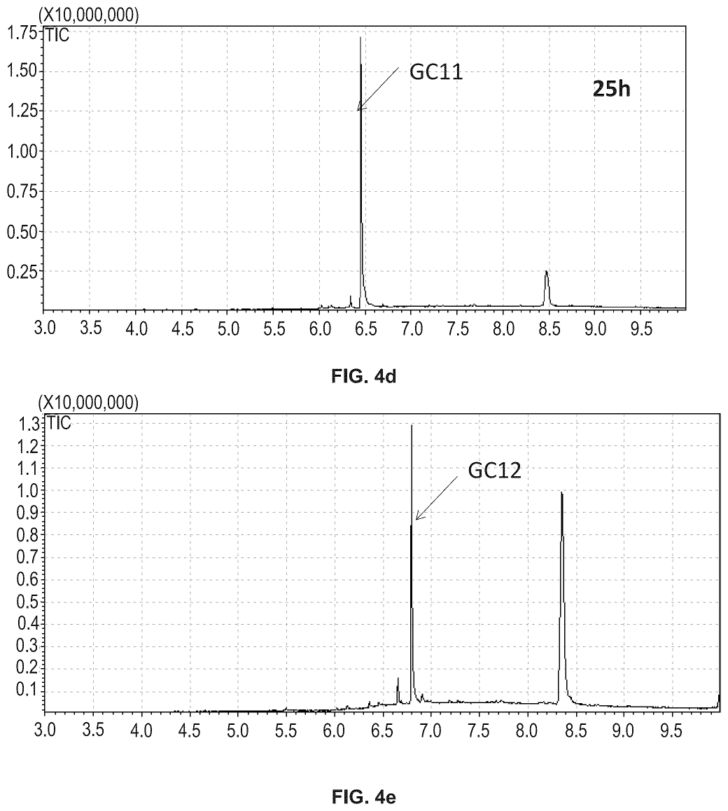

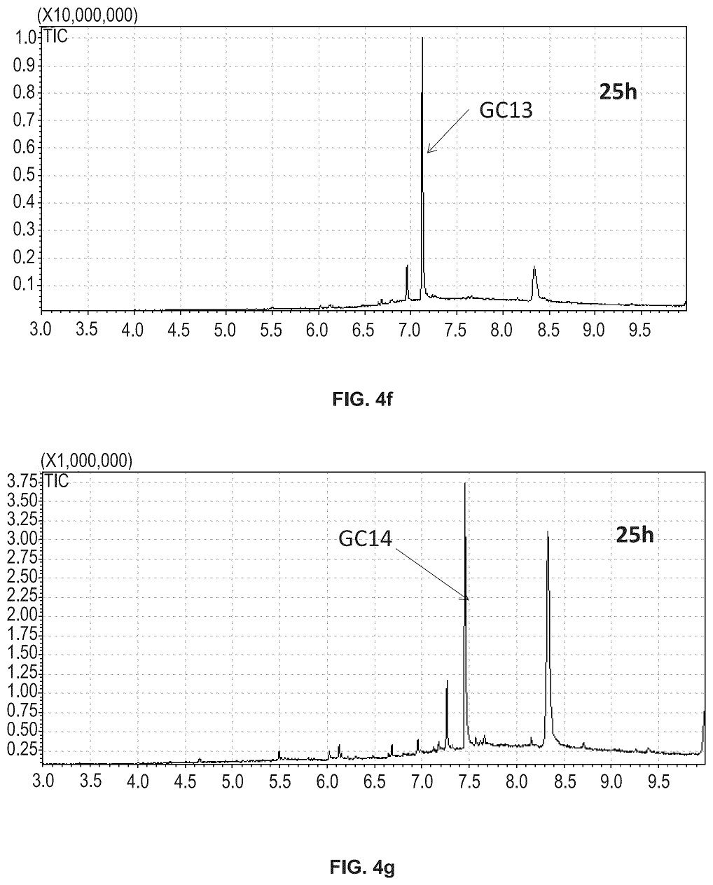

The procedures described in Example 2 were performed using an E. coli culture overexpressing Umbelopsis isabellina P450. Specifically, samples of the E. coli culture were taken and acidified 25 hours after adding the fatty acid substrate. FIGS. 4a-4g show the production of various gamma-lactones from the corresponding carboxylic acid: (a) γ-undecalactone (GC11) from undecylic acid (undecanoic acid, C11), and (b) γ-undecenolactone (GC11′) from undecylenic acid (undecenoic acid, C11:1) retention time min.

FIGS. 4a-4g shows the production of various gamma-lactones from the corresponding carboxylic acid: (a) γ-hexalactone (GC6) from hexanoic acid (C6), (b) γ-heptalactone (GC7) from enanthic acid (heptanoic acid, C7), (c) γ-decalactone (GC10) from capric acid (decanoic acid, C10), (d) γ-undecalactone (GC11) from undecylic acid (undecanoic acid, C11), (e) γ-dodecalactone (GC12) from lauric acid (dodecanoic acid, C12), (f) γ-tridecalactone (GC13) from tridecylic acid (tridecanoic acid, C13), and (g) γ-tetradecalactone (GC14) from myristic acid (tetradecanoic acid, C14).

As shown in FIG. 4a to FIG. 4g, GC6, GC7, GC10, GC11, GC12, GC13, and GC14 γ-lactones were effectively produced from the corresponding C6, C7, C10, C11, C12, C13, and C14 carboxylic acids in a heterologous system that overexpressed Umbelopsis isabellina cytochrome P450 (SEQ ID NO: 5). Notably, the GC6, GC7, GC10, GC11 and GC12 peaks match the corresponding standards from Sigma-Aldrich (St Louis, MO) in terms of both retention times and mass spectra. The standards for γ-tridecalactone (GC13) and γ-tetradecalactone (GC14) are not commercially available. Nevertheless, it was noted that the putative GC13 and GC14 peaks have retention times longer than that of GC12.

Under the reaction conditions described, about 42 mg/L of GC6 was produced 25 hours after adding 1 g/L of C6 to the E. coli growing cell culture.

In addition to growing cells, resting cells were also investigated for their potential use in the bioconversion of fatty acids into gamma-lactones. In this experiment, an overnight culture was used to inoculate liquid SOC medium (3%) containing 100 mg/L of carbenicillin. The culture was first grown at 37° C. to an OD600 of 0.8 and cooled down to 16° C. Then, 1 mM of IPTG was added to induce protein expression. After 16h of incubation at 16° C., the culture was harvested and cell pellets were collected by centrifugation and stored at −80° C. until use. In a typical bioconversion experiment, the frozen cells were re-suspended in 100 mM K-Pi buffer, pH7 at a cell concentration of 100 g/L fresh weight. Then, 1 g/L of C6 was added and the mixture was incubated at 30° C. with shaking at 250 rpm. Samples were taken 3h later. The titer for GC6 was 198 mg/L.

Sequences of Interest:
Mucor ambiguus cytochrome P450
Amino acid sequence of the GAN03094.1 gene
(SEQ ID NO: 1):
MTKYAHDQIPGPEPHYLLGNVPDIFPDSLGNLIKLHDKYGPIVHLSMGGHELLSVDDPAVLE
TICEDNEYFTKEIESVYSDLAILNGRGLVTTSTADPDWQLGHKLIMNAFSARAMKAYHYKMG
ESISELCEIMDSFAKSGEDFDVSRWFIALALESIGKIGFDYDFDLLKDPNAPRHPFTVALAY
VQSMIMKRASTLSWLKWYQTTTNVRFHRDLQTLRGTVEEVLKDRREHPHTEADQSDLLDFMI
KAESKEGEKLNDSLIRDNIITFLSAGHNTTSAFLSWTMLELCKHPEVVENIKQEIANCGIKA
GEVPTPEQVKECKYLDLVIKESLRIHPPITSILKYCKKDATVKASNGDEYDIKAGQLLQVNI
NALHHNPKVWEDPDVFNPDRFSGDTDNLPNTAWLTFSTGPRACIGRQFALQEGKLALVMILS
RFHFKMDDPSQKIGYAVVVSTKPVGFFAKIESSQLPEPTEEIVVTKRRESKAVPQEKVKPAE
FPLPPVTFLFGTQTNTSEEYARKLSGQAKEMGFKEVTVQDLDDWKLVKGEAIAKAQHDADAP
SSEDDVKVSELVVVVTATYNGFPPDDANEFAKWLDERTKDSEATKNNMLSGMLYAVFGCGNR
DWTSTFQKFPKKVDSGFELLGGERLLPAGEGDASDDIDGDESLWSASFWTALMQRYGQSSSG
KNADIMSNNGPAADPSQDFTLEFINIVKEKVKTEQAALNCNQLETVATIVENRELQHTEKSH
RSTRHIQVQFDKSVDGKPLYEAGDHLEVVPVNEDRLVEIIATNLGLVLDSVFEVKDLDIKNL
SPRSVAANIKGPCTIRNALKYYADLTGPPTRFSLSILSKQLKDSRPDIAERLQKALQPGKET
ERLKEFLASHRTLIDIIQAFKIKELNFKEFISSVNCIVPRKYSISSGPLEHPFDPSISVGVV
DTVGGPDGNTHYFGLASGYLSHQEPGTKINAQIKACKSTFRLPDDPSTPVIFIAAGTGFSPF
RGFLQERHAKGLKSSKKNSNGESSECYMFFGCRHPDQDFIYKEEFDAYLEDGTITELYTTFS
RSGEVVKYVQHALLKHANLLYKLMEESNAKVYICGSAGSMAKDVKRTWERLLVQMSGVSESE
AAEQIQAWVDEGKYNEDVWGT
Nucleotide sequence of GAN03094.1, codon-optimized
for E. coli genome (SEQ ID NO: 2):
ATGACCAAATATGCCCATGATCAGATTCCGGGTCCTGAACCGCATTATCTGCTGGGTAATGT
TCCGGATATTTTTCCGGATAGCCTGGGCAATCTGATTAAACTGCATGATAAATATGGTCCGA
TTGTGCATCTGAGCATGGGTGGTCATGAACTGCTGAGCGTTGATGATCCGGCAGTTCTGGAA
ACCATTTGTGAAGATAATGAATATTTCACCAAAGAAATCGAAAGCGTGTATAGCGATCTGGC
AATTCTGAATGGTCGTGGTCTGGTTACCACCAGTACCGCAGATCCGGATTGGCAGCTGGGTC
ATAAACTGATTATGAATGCATTTAGCGCACGTGCCATGAAAGCCTATCACTATAAAATGGGT
GAAAGCATTAGCGAACTGTGCGAAATTATGGATAGCTTTGCAAAAAGCGGTGAGGATTTTGA
TGTTAGCCGTTGGTTTATTGCACTGGCACTGGAAAGCATTGGTAAAATCGGTTTCGATTATG
ATTTCGACCTGCTGAAAGATCCGAATGCACCGCGTCATCCGTTTACCGTTGCGCTGGCCTAT
GTTCAGAGTATGATCATGAAACGTGCAAGCACCCTGAGCTGGCTGAAATGGTATCAGACCAC
CACCAATGTTCGTTTTCATCGTGATCTGCAGACCCTGCGTGGCACCGTTGAAGAAGTTCTGA
AAGACCGTCGTGAACATCCGCATACCGAAGCAGATCAGAGCGATCTGCTGGATTTTATGATT
AAAGCCGAAAGCAAAGAAGGCGAGAAACTGAACGATAGCCTGATTCGTGATAACATCATTAC
CTTTCTGAGCGCAGGTCATAATACCACCTCAGCATTTCTGAGCTGGACCATGCTGGAACTGT
GTAAACATCCGGAAGTTGTCGAAAACATCAAACAAGAAATTGCCAACTGCGGTATTAAAGCG
GGTGAAGTTCCGACACCGGAACAGGTTAAAGAATGTAAATATCTGGACCTGGTGATCAAAGA
AAGCCTGCGTATTCATCCGCCTATTACCAGCATTCTGAAATACTGTAAAAAAGACGCAACCG
TGAAAGCCAGCAATGGTGATGAATATGATATTAAAGCAGGTCAGCTGCTGCAGGTTAACATT
AATGCACTGCATCATAACCCGAAAGTTTGGGAAGATCCTGATGTTTTTAACCCGGATCGTTT
TAGCGGTGATACCGATAATCTGCCGAATACCGCATGGCTGACCTTTAGCACCGGTCCGCGTG
CATGTATTGGTCGTCAGTTTGCACTGCAAGAAGGTAAACTGGCCCTGGTTATGATTCTGAGC
CGTTTTCATTTCAAAATGGATGATCCGAGCCAGAAAATTGGTTATGCAGTTGTTGTTAGCAC
CAAACCGGTTGGTTTTTTTGCCAAAATTGAAAGCAGCCAGCTGCCGGAACCGACCGAAGAAA
TTGTTGTTACCAAACGTCGTGAAAGTAAAGCAGTTCCGCAAGAAAAAGTTAAACCGGCAGAA
TTTCCGCTGCCTCCGGTGACCTTTCTGTTTGGCACCCAGACCAATACCAGCGAAGAATATGC
ACGTAAACTGAGCGGTCAGGCAAAAGAAATGGGTTTTAAAGAAGTTACCGTCCAGGATCTGG
ATGATTGGAAACTGGTTAAAGGTGAAGCAATTGCAAAAGCACAGCATGATGCCGATGCACCG
AGCAGCGAAGATGATGTTAAAGTGAGCGAACTGGTTGTTGTTGTGACCGCAACCTATAATGG
TTTTCCGCCTGATGATGCAAACGAATTTGCAAAATGGCTGGATGAACGTACCAAAGATAGCG
AAGCAACCAAAAATAACATGCTGAGCGGTATGCTGTATGCAGTGTTTGGTTGTGGTAATCGT
GATTGGACCAGCACCTTTCAGAAATTCCCGAAAAAAGTTGATAGCGGCTTTGAACTGTTAGG
TGGTGAACGTCTGCTGCCAGCCGGTGAAGGTGATGCAAGTGATGATATTGATGGTGATTTTA
GCCTGTGGTCTGCCAGCTTTTGGACCGCACTGATGCAGCGTTATGGTCAGAGCAGCAGCGGT
AAAAATGCAGATATTATGAGCAATAATGGTCCGGCAGCAGATCCGAGTCAGGATTTTACCCT
GGAATTTATCAACATCGTGAAAGAGAAGGTCAAAACCGAACAGGCAGCACTGAATTGTAATC
AGCTGGAAACCGTTGCAACCATTGTTGAAAATCGCGAACTGCAGCATACAGAAAAAAGCCAT
CGTAGCACCCGTCATATTCAGGTTCAGTTTGATAAAAGCGTGGATGGTAAACCGCTGTATGA
AGCCGGTGATCATCTGGAAGTGGTTCCGGTTAATGAAGATCGTCTGGTTGAAATTATTGCCA
CCAATCTGGGTTTAGTTCTGGATAGCGTTTTTGAGGTGAAAGACCTGGATATTAAGAATCTG
AGTCCGCGTAGCGTTGCAGCAAACATTAAAGGTCCGTGTACCATTCGTAATGCCCTGAAATA
TTACGCAGATCTGACCGGTCCTCCGACACGTTTTAGTCTGAGCATTCTGTCAAAACAGCTGA
AAGACAGCCGTCCTGATATTGCAGAACGCCTGCAGAAAGCACTGCAGCCTGGTAAAGAAACC
GAACGTCTGAAAGAATTTCTGGCAAGTCATCGTACCCTGATCGATATTATTCAGGCCTTCAA
AATCAAAGAACTGAACTTCAAAGAGTTTATCAGCAGCGTTAATTGCATCGTTCCGCGTAAAT
ATAGCATTAGCAGTGGTCCGCTGGAACATCCGTTTGATCCGAGTATTAGCGTTGGTGTTGTT
GATACCGTTGGTGGTCCGGATGGTAATACCCATTATTTTGGTCTGGCAAGCGGTTATCTGAG
CCATCAAGAACCGGGTACAAAAATCAATGCACAGATTAAAGCATGCAAGAGTACCTTTCGTC
TGCCGGATGATCCTAGCACACCGGTTATCTTTATTGCAGCAGGCACCGGTTTTAGCCCGTTT
CGTGGTTTTCTGCAAGAACGTCATGCAAAAGGTCTGAAAAGCAGCAAAAAAAACAGCAATGG
CGAAAGCAGCGAGTGCTATATGTTTTTTGGCTGTCGTCATCCGGATCAGGATTTCATCTATA
AAGAAGAATTTGACGCCTACCTGGAAGATGGCACCATTACAGAACTGTATACCACCTTTAGC
CGTAGCGGTGAAGTTGTTAAATATGTTCAGCACGCACTGCTGAAACATGCAAATCTGCTGTA
CAAACTGATGGAAGAAAGCAACGCCAAAGTGTATATTTGTGGTAGCGCAGGTAGCATGGCAA
AAGATGTTAAACGTACCTGGGAGCGCCTGCTGGTTCAGATGAGCGGTGTTAGCGAAAGCGAA
GCAGCAGAGCAGATTCAGGCATGGGTTGATGAAGGCAAATATAACGAAGATGTTTGGGGCAC
CTAA
Basidiobolus meristosporus cytochrome P450
Amino acid sequence of the ORX85448.1 gene
(SEQ ID NO: 3):
MSSQSDEIPGPKPHILLGNVQDIIPDTIGNHRRLHDEYGPVMRLYLGGHDFVSICDPEVIQS
ITGEGDFTKEIYSAYEDLAILSGRGLVTTATKDPDWILAHKLLMPAFSARAMKAYHDIMGKC
ILDLLKIMESYQECGEAIDMSRWMISLALESIGVIGFGYNFNLLDDKDSERHPFSVALNYVQ
SMIMKRTNSVTWTKWLQTTANVRFRRDLATLRDTVDDVLKQRRLNPPSEDARRDLLDFMLAA
ESKQGEKLDDNLIRDEIITFLSAGHNTTSSFLSWTFFELARNPDVEQKILQEIVNAGIKPGE
IPSTEQVAKCKYIDMVIKESLRFHSPIPLVIKYCQNDCTIKTSEGKEYEIKRGQLAQIQISA
VHKDPKLWENPNVFDPERFNPEKEADRHPCAWIPFSDGPRACIGRQFSLQEGKLALIMLLCK
FRFRLLDETKEVGYQIIVSLKPVDLIMKVLPAELPSPNAGSDTSSVKDTVKPQLKPKENEVL
EHARFPLPPVTFLYGTQTGTSEEYARKLSGQAKEFGFTDIAVAELDDWEVVKNNRIPSNKGQ
SSPSDEDGTKVSQLAVIVTATYNGYPPDNALKFDSWLTDITKDQKNQLEGLLYAVFGCGNKQ
WQSTFQAFPKKVDASIELLGAERLVPAGAGNADQDIDGDFTNWSASFWAALMQKYGRGASDK
DADLMTTSGPVADPSNDFTLEFLPLGGDQAAMEAALGNRNSDPGAQVAILENKELQDVEKSG
RSTRHIVVECPPASPGSGKKASYRAGDHLEIKPYNDDQLVENIAIGFGYVLDSVFQIKDCKI
TNLSPRSLAANIIGPCTVRNALTYFADLSGPPTRYTLTVMAKQLEKTRPDVAERLLHALQPG
KETPRLREFLSTHRTILDIQRAFKIKELSFKEFLSSVNVIVPRRYSISSGPLEHPNEVSVTV
GVVKDIGGADNNTDYYGLASGYLMRCPIGSKIDAKIKPCKNNFRLPENEQTPVIFICAGTGF
APFRGFLQERHAKGWKSKEKGGSSDAYLFFGCRHPDHDFIYQKELEEYLEDGTLTKLYTTFS
RYNQTKKYVQHLLLTHAQLLENLIMNENANIYVCGAGRGMAHDVRRTFERLAVQVAGMSEPE
AVDAISHMVDAERYNEDVWG
Nucleotide sequence of ORX85448.1, codon-
optimized for E. coli genome (SEQ ID NO: 4):
ATGAGCAGCCAGAGCGATGAAATTCCGGGCCCGAAACCGCATATTCTGCTGGGTAATGTTCA
GGATATTATTCCGGATACCATTGGCAATCATCGCCGTCTGCATGATGAATATGGCCCGGTTA
TGCGTCTGTATCTGGGTGGTCATGATTTTGTGAGCATTTGTGATCCGGAAGTGATTCAGAGC
ATTACCGGCGAAGGCGATTTTACCAAAGAAATCTATAGCGCATACGAAGATCTGGCAATTCT
GAGCGGCCGTGGCCTGGTTACCACCGCAACCAAAGATCCGGATTGGATTCTGGCCCATAAAC
TGCTGATGCCGGCCTTTAGTGCCCGCGCAATGAAAGCCTATCATGATATTATGGGTAAATGT
ATTCTGGATCTGCTGAAAATTATGGAAAGCTATCAGGAATGTGGTGAAGCCATTGATATGAG
CCGTTGGATGATTAGCCTGGCACTGGAAAGTATTGGCGTTATTGGTTTTGGCTATAATTTTA
ATCTGCTGGATGATAAAGACAGCGAACGCCATCCGTTTAGCGTTGCACTGAATTATGTGCAG
AGTATGATTATGAAACGCACCAATAGCGTTACCTGGACCAAATGGCTGCAGACCACCGCCAA
TGTGCGTTTTCGTCGCGATCTGGCCACCCTGCGCGATACCGTGGATGATGTGCTGAAACAGC
GTCGTCTGAATCCGCCGAGCGAAGATGCCCGCCGCGATCTGCTGGATTTTATGCTGGCCGCC
GAAAGCAAACAGGGCGAAAAACTGGATGATAATCTGATTCGCGATGAAATTATTACCTTTCT
GAGTGCAGGCCATAATACCACCAGTAGTTTTCTGAGTTGGACCTTTTTCGAACTGGCCCGCA
ATCCGGATGTGGAACAGAAAATTCTGCAGGAAATTGTGAATGCCGGCATTAAGCCGGGTGAA
ATTCCGAGTACCGAACAGGTGGCCAAATGTAAATATATTGATATGGTTATCAAGGAGAGCCT
GCGCTTTCATAGCCCGATTCCGCTGGTTATTAAGTATTGTCAGAATGATTGCACCATTAAGA
CCAGTGAAGGTAAAGAATATGAAATTAAGCGCGGCCAGCTGGCCCAGATTCAGATTAGCGCC
GTGCATAAAGATCCGAAACTGTGGGAAAATCCGAATGTGTTTGATCCGGAACGCTTTAATCC
GGAAAAAGAAGCAGATCGTCATCCGTGTGCATGGATTCCGTTTAGCGATGGTCCGCGTGCCT
GCATTGGTCGCCAGTTTAGCCTGCAGGAAGGTAAACTGGCCCTGATTATGCTGCTGTGCAAA
TTTCGTTTTCGTCTGCTGGATGAAACCAAAGAAGTGGGTTATCAGATTATTGTGAGCCTGAA
ACCGGTGGATCTGATTATGAAAGTGCTGCCGGCAGAACTGCCGAGTCCGAATGCCGGCAGCG
ATACCAGTAGTGTGAAAGATACCGTTAAACCGCAGCTGAAACCGAAAGAAAATGAAGTGCTG
GAACATGCCCGTTTTCCGCTGCCGCCGGTTACCTTTCTGTATGGTACCCAGACCGGCACCAG
TGAAGAATATGCACGCAAACTGAGTGGTCAGGCAAAAGAATTTGGCTTTACCGATATTGCCG
TGGCAGAACTGGATGATTGGGAAGTTGTTAAAAATAATCGTATCCCGAGTAATAAGGGTCAG
AGTAGTCCGAGCGATGAAGATGGCACCAAAGTGAGCCAGCTGGCTGTTATTGTTACCGCAAC
CTATAATGGTTATCCGCCGGATAATGCACTGAAATTTGATAGCTGGCTGACCGATATTACCA
AAGATCAGAAAAATCAGCTGGAAGGTCTGCTGTATGCCGTGTTTGGTTGTGGCAATAAGCAG
TGGCAGAGCACCTTTCAGGCATTTCCGAAAAAAGTGGATGCCAGTATTGAACTGCTGGGCGC
AGAACGTCTGGTGCCGGCAGGCGCAGGCAATGCCGATCAGGATATTGATGGTGACTTTACCA
ATTGGAGTGCCAGCTTTTGGGCCGCACTGATGCAGAAATATGGCCGCGGCGCCAGTGATAAA
GATGCCGATCTGATGACCACCAGTGGCCCGGTGGCCGATCCGAGTAATGATTTTACCCTGGA
ATTTCTGCCGCTGGGTGGTGACCAGGCAGCAATGGAAGCAGCCCTGGGTAATCGTAATAGCG
ATCCGGGCGCACAGGTGGCCATTCTGGAAAATAAGGAACTGCAGGATGTTGAAAAAAGTGGC
CGTAGTACCCGCCATATTGTTGTTGAATGCCCGCCGGCCAGCCCGGGCAGTGGTAAAAAAGC
AAGTTATCGCGCCGGTGACCATCTGGAAATTAAGCCGTATAATGATGATCAGCTGGTGGAAA
ATATTGCCATTGGCTTTGGCTATGTTCTGGATAGTGTTTTTCAGATTAAGGATTGCAAAATC
ACCAATCTGAGTCCGCGCAGTCTGGCCGCCAATATTATTGGTCCGTGCACCGTTCGCAATGC
CCTGACCTATTTTGCCGATCTGAGTGGTCCGCCGACCCGCTATACCCTGACCGTGATGGCAA
AACAGCTGGAAAAAACCCGCCCGGATGTGGCAGAACGTTTACTGCATGCACTGCAGCCGGGC
AAAGAAACCCCGCGCCTGCGCGAATTTCTGAGTACCCATCGTACCATTCTGGATATTCAGCG
CGCCTTTAAAATTAAGGAACTGAGCTTTAAAGAGTTCCTGAGCAGTGTTAATGTTATTGTTC
CGCGCCGCTATAGTATTAGCAGCGGCCCGCTGGAACATCCGAATGAAGTGAGTGTGACCGTG
GGTGTTGTTAAAGATATTGGCGGTGCAGATAATAATACCGATTATTATGGTCTGGCCAGTGG
TTATCTGATGCGCTGCCCGATTGGTAGTAAAATTGATGCCAAAATTAAGCCGTGCAAAAATA
ATTTTCGCCTGCCGGAAAATGAACAGACCCCGGTTATTTTTATTTGCGCCGGCACCGGCTTT
GCCCCGTTTCGTGGTTTTCTGCAGGAACGCCATGCCAAAGGTTGGAAAAGTAAAGAAAAAGG
CGGTAGTAGCGATGCATATCTGTTTTTCGGCTGTCGTCATCCGGATCATGATTTTATCTATC
AGAAAGAACTGGAAGAATACCTGGAAGATGGTACCCTGACCAAACTGTATACCACCTTTAGC
CGCTATAATCAGACCAAAAAATATGTGCAGCATCTGCTGCTGACCCATGCCCAGCTGCTGTT
TAATCTGATTATGAATGAAAACGCAAACATCTATGTGTGTGGTGCAGGCCGTGGCATGGCCC
ATGATGTGCGCCGCACCTTTGAACGCCTGGCAGTTCAGGTTGCAGGCATGAGCGAACCGGAA
GCCGTGGATGCCATTAGTCACATGGTTGATGCCGAACGCTATAATGAAGATGTTTGGGGTTA
A
Umbelopsis isabellina cytochrome P450
Amino acid sequence (SEQ ID NO: 5):
MTVYESDKIPGPEPRVLLGNIPDVYPDFVGNITQLHEKYGPVMRLYLGGHDYVSVCDPDCLQ
TTHKDGEYFTKEIQSTYEDLAILNGRGLVTTATKDPDWILGHKLLMPAFSARAMKAYHYKMG
DTIKDLLNIIESFQKSGEDFDVSRWMIALALESIGNIGFDYDFNLLKDPNAERHPFTVALSY
VQSMIMKRSASVSWMKWMKTSANARFQRDLGTLRNTLDSVLKERHEHPHSEDQQSDLLDFMI
SASTKEGDKLDDKIIRDNVMTFLSAGHNTTSSFLSWTILELCRHPEVAATIRQELANAGVQP
GEIPTPEQVNACKYLDLVIKESLRMHPPIVAVLKYCKKDCTIKAGTTGDEYKIKAGQLLQSN
INALHHSTKVWDEPMVFNPDRFADAELHPNAWMPFSDGPRACIGRQFSLQEGKLALVMMLSK
FNFSMEDPSQKIGYEIIVAIKPVGLMVKVTPAELPEPTEEIVVTORRESKAEPQESLKPAEF
PLPPVTILYGTQTNTSEEYAKKLSGQAKEFGFKTIKVDDLDNWKLLNGGKLTKLNKDQSAPS
SGDDVKVSELVVVVTATYNGNPPDNAMKFDEWLSKKTESIEDTKSNELEGILYAVFGCGNRD
WSSTFQKFPTAVDTGLELLGGERLLPAGVGDASDDIDGDFSEWSANFWSTLMQRYGQSSSGK
NADIMTSTAPLADPSKDFNLEFLPVHKNKELVTQANENRNQRGKTVTIKENRELQNIEKSHR
STKHIEVQFDKSEDGKPLYIAGDHLEITPVNKEELVELVAVNLGLVLDSVFQIQMNEVDISH
LSPRSLAANIKGPCTIRNALKYYADLTGPPTRYTLSVLGKQLEKTRPEIAKRLQEALQPGKE
TPRLKEFLSTHRTFVDIMKAFNIKELNFKEFLSSVNCIVPRKYSISSGPTEHPFDPSVSIGI
VRDIGGPDGKTEYRGLASGYLDTLKPGSQVNAQIKDCKSTFRLPDDGSTPVIFICAGTGMSP
FRGFLQERHAQGLKSSKKGGSSEAYMFFGCRHPDQDFIYKDELQSYVEDGTLTELYTTFSRS
NQVVKYVQHSLLQHAQMLYELMVDHNAKVYVCGSAGSMAKDVKRTWERITVQMSGMSEPEAE
DLLKEWSDKGKYNEDVWGT
Nucleotide sequence, codon-optimized for
E. coli genome (SEQ ID NO: 6):
ATGACCGTGTATGAAAGCGATAAAATTCCGGGTCCGGAACCGCGCGTTCTGCTGGGTAATAT
TCCGGATGTTTATCCGGATTTTGTTGGCAATATTACCCAGCTGCATGAAAAATATGGTCCGG
TTATGCGTCTGTATCTGGGCGGCCATGATTATGTGAGTGTTTGTGATCCGGATTGTCTGCAG
ACCACCCATAAAGATGGTGAATATTTTACCAAAGAGATCCAGAGCACCTATGAAGATCTGGC
CATTCTGAATGGCCGTGGCCTGGTGACCACCGCCACCAAAGATCCGGATTGGATTCTGGGCC
ATAAACTGCTGATGCCGGCATTTTCAGCCCGCGCCATGAAAGCCTATCATTATAAAATGGGT
GACACCATTAAGGATCTGCTGAATATTATTGAAAGTTTTCAGAAGTCCGGTGAAGATTTTGA
TGTTAGCCGTTGGATGATTGCCCTGGCACTGGAAAGCATTGGCAATATTGGTTTTGATTATG
ATTTCAACCTGCTGAAAGATCCGAATGCCGAACGTCATCCGTTTACCGTTGCCCTGAGTTAT
GTTCAGAGTATGATTATGAAACGCAGTGCAAGCGTTAGCTGGATGAAATGGATGAAAACCAG
TGCCAATGCCCGCTTTCAGCGTGATCTGGGTACCCTGCGCAATACCCTGGATAGTGTGCTGA
AAGAACGCCATGAACATCCGCATAGCGAAGATCAGCAGAGCGATCTGCTGGATTTTATGATT
AGTGCCAGTACCAAAGAAGGCGATAAACTGGATGATAAAATTATTCGCGATAACGTTATGAC
CTTTCTGAGCGCAGGTCATAATACCACCAGCAGTTTTCTGAGTTGGACCATTCTGGAACTGT
GCCGTCATCCGGAAGTTGCCGCAACCATTCGTCAGGAACTGGCAAATGCCGGTGTGCAGCCG
GGTGAAATTCCGACCCCGGAACAGGTGAATGCCTGTAAATATCTGGATCTGGTTATTAAGGA
AAGCCTGCGTATGCATCCGCCGATTGTGGCAGTTCTGAAATATTGTAAAAAGGATTGTACCA
TCAAGGCCGGTACCACCGGTGACGAATATAAAATTAAGGCAGGCCAGCTGCTGCAGAGCAAT
ATTAATGCACTGCATCATAGCACCAAAGTTTGGGATGAACCGATGGTTTTTAATCCGGATCG
TTTTGCCGATGCAGAACTGCATCCGAATGCCTGGATGCCGTTTAGTGATGGTCCGCGTGCCT
GTATTGGCCGTCAGTTTAGCCTGCAGGAAGGCAAACTGGCACTGGTTATGATGCTGAGCAAA
TTCAATTTTAGCATGGAAGATCCGAGCCAGAAAATTGGCTATGAAATTATTGTGGCCATTAA
GCCGGTGGGCCTGATGGTGAAAGTGACCCCGGCCGAACTGCCGGAACCGACCGAAGAAATTG
TTGTTACCCAGCGCCGTGAAAGTAAAGCAGAACCGCAGGAAAGTCTGAAACCGGCCGAATTT
CCGCTGCCGCCGGTTACCATTCTGTATGGCACCCAGACCAATACCAGCGAAGAATATGCAAA
AAAGCTGAGCGGCCAGGCCAAAGAATTTGGTTTTAAAACCATTAAGGTGGATGATCTGGATA
ATTGGAAACTGCTGAATGGCGGTAAACTGACCAAACTGAATAAGGATCAGAGTGCACCGAGT
AGTGGTGACGATGTGAAAGTGAGTGAACTGGTGGTGGTTGTTACCGCAACCTATAATGGCAA
TCCGCCGGATAATGCAATGAAATTTGATGAATGGCTGAGTAAAAAGACCGAAAGCATTGAAG
ATACCAAAAGCAATGAACTGGAAGGCATTCTGTATGCAGTTTTTGGCTGCGGCAATCGTGAT
TGGAGCAGTACCTTTCAGAAATTTCCGACCGCAGTGGATACCGGTCTGGAACTGCTGGGCGG
CGAACGTCTGCTGCCGGCAGGCGTTGGCGATGCCAGTGATGATATTGATGGCGATTTTAGCG
AATGGAGCGCAAATTTTTGGAGTACCCTGATGCAGCGTTATGGTCAGAGCAGTAGCGGCAAA
AATGCAGATATTATGACCAGCACCGCACCGCTGGCAGATCCGAGTAAAGATTTTAATCTGGA
ATTTCTGCCGGTGCATAAAAATAAGGAACTGGTGACCCAGGCAAATGAAAATCGCAATCAGC
GCGGTAAAACCGTGACCATTAAGGAAAATCGCGAACTGCAGAATATTGAAAAAAGCCATCGT
AGTACCAAACATATTGAAGTTCAGTTTGATAAGAGCGAAGATGGTAAACCGCTGTATATTGC
CGGTGACCATCTGGAAATTACCCCGGTTAATAAGGAAGAACTGGTTGAACTGGTGGCCGTTA
ATCTGGGTCTGGTGCTGGATAGCGTGTTTCAGATTCAGATGAATGAAGTTGATATTAGTCAC
CTGAGCCCGCGCAGTCTGGCAGCAAATATTAAGGGCCCGTGCACCATTCGCAATGCACTGAA
ATATTATGCAGATCTGACCGGTCCGCCGACCCGTTATACCCTGAGTGTGCTGGGTAAACAGC
TGGAAAAAACCCGTCCGGAAATTGCCAAACGTCTGCAGGAAGCCCTGCAGCCGGGCAAAGAA
ACCCCGCGCCTGAAAGAATTTCTGAGTACCCATCGTACCTTTGTTGATATTATGAAAGCATT
CAATATCAAGGAGCTGAATTTTAAAGAGTTCCTGAGCAGTGTGAATTGTATTGTGCCGCGCA
AATATAGCATTAGTAGTGGTCCGACCGAACATCCGTTTGATCCGAGCGTGAGCATTGGTATT
GTTCGCGATATTGGTGGTCCGGATGGCAAAACCGAATATCGCGGTCTGGCAAGCGGCTATCT
GGATACCCTGAAACCGGGTAGTCAGGTGAATGCGCAGATTAAGGATTGTAAAAGTACCTTTC
GCCTGCCGGATGATGGTAGCACCCCGGTTATTTTTATTTGCGCCGGTACCGGCATGAGCCCG
TTTCGTGGCTTTCTGCAGGAACGCCATGCCCAGGGTCTGAAAAGCAGTAAAAAAGGTGGCAG
TAGTGAAGCATATATGTTTTTCGGTTGCCGCCATCCGGATCAGGATTTTATCTATAAAGATG
AACTGCAGAGTTACGTTGAAGATGGTACCCTGACCGAACTGTATACCACCTTTAGTCGTAGT
AATCAGGTGGTTAAATATGTGCAGCATAGCCTGCTGCAGCATGCACAGATGCTGTATGAACT
GATGGTGGATCATAATGCAAAAGTTTATGTTTGTGGCAGCGCCGGTAGCATGGCAAAAGATG
TTAAACGCACCTGGGAACGCATTACCGTTCAGATGAGTGGCATGAGCGAACCGGAAGCCGAA
GATCTGCTGAAAGAATGGAGCGATAAAGGCAAATATAATGAAGATGTGTGGGGCACCTAA

1. A method for producing a C4-C20 gamma lactone, the method comprising:
providing a reaction mixture comprising a recombinant CYP450 protein having carboxylic acid 4-hydroxylase activity and a C4-C20 carboxylic acid substrate to provide a 4-hydroxy C4-C20 carboxylic acid; and
subjecting the 4-hydroxy C4-C20 carboxylic acid to acidic conditions to produce the C4-C20 gamma lactone;
wherein the recombinant CYP450 protein comprises an amino acid sequence having at least 80% identity to SEQ ID NO: 3, or SEQ ID NO: 5.

2. The method of claim 1, wherein the recombinant CYP450 protein is expressed by a transformed host cell in the reaction mixture.

3. The method of claim 2, wherein the transformed host cell is selected from a bacterial cell and a yeast cell.

4. (canceled)

5. The method of claim 1, wherein the recombinant CYP450 protein (a) comprises an amino acid sequence having at least 85% identity to SEQ ID NO: 3; (b) comprises an amino acid sequence having at least 90% identity to SEQ ID NO: 3; (c) comprises an amino acid sequence having at least 95% identity to SEQ ID NO: 3; or (d) comprises the amino acid sequence of SEQ ID NO: 3.

6. The method claim 1, wherein the recombinant CYP450 protein (a) comprises an amino acid sequence having at least 85% identity to SEQ ID NO: 5; (b) comprises an amino acid sequence having at least 90% identity to SEQ ID NO: 5; (c) comprises an amino acid sequence having at least 95% identity to SEQ ID NO: 5; or (d) comprises the amino acid sequence of SEQ ID NO: 5.

7. The method of claim 1, wherein the C4-C20 carboxylic acid substrate comprises a C4-C20 carboxylic acid; a salt of a C4-C20 carboxylic acid; an ester of a C4-C20 carboxylic acid; a mono-, di- or triglyceride of a C4-C20 carboxylic acid; or a combination thereof.

8. The method of claim 1, wherein the C4-C20 carboxylic acid comprises a straight-chain C4-C20 carboxylic acid, a branched C4-C20 carboxylic acid, a saturated C4-C20 carboxylic acid, an unsaturated C4-C20 carboxylic acid, or a combination thereof.

9. (canceled)

10. The method of claim 5 for producing a C4-C20 gamma lactone selected from γ-undecalactone and γ-undecenolactone, wherein the C4-C20 carboxylic acid substrate is selected from undecanoic acid and undecenoic acid.

11. The method of claim 6 for producing a C4-C20 gamma lactone selected from γ-hexalactone, γ-heptalactone, γ-decalactone, γ-undecalactone, γ-dodecalactone, γ-tridecalactone, and γ-tetradecalactone, wherein the C4-C20 carboxylic acid substrate is selected from hexanoic acid, heptanoic acid, decanoic acid, undecanoic acid, dodecanoic acid, tridecanoic acid, and tetradecanoic acid.

12. A method for producing a C4-C20 gamma lactone, the method comprising:
incubating a transformed host cell comprising a sequence encoding a CYP450 protein having carboxylic acid 4-hydroxylase activity with a C4-C20 carboxylic acid substrate to provide a 4-hydroxy C4-C20 carboxylic acid; and
subjecting the 4-hydroxy C4-C20 carboxylic acid to acidic conditions to produce the C4-C20 gamma lactone;
wherein the sequence encoding the CYP450 protein comprises a nucleotide sequence having
at least 80% identity to SEQ ID NO: 2, SEQ ID NO: 4, or SEQ ID NO: 6.

13. The method of claim 12, wherein the transformed host cell is selected from a bacterial cell or a yeast cell.

14. The method of claim 13, wherein the nucleotide sequence has at least 80% identity to SEQ ID NO: 2 and the CYP450 protein (a) comprises an amino acid sequence having at least 85% identity to SEQ ID NO: 1; (b) comprises an amino acid sequence having at least 90% identity to SEQ ID NO: 1; (c) comprises an amino acid sequence having at least 95% identity to SEQ ID NO: 1; or (d) comprises the amino acid sequence of SEQ ID NO: 1.

15. The method of claim 13, wherein the nucleotide sequence has at least 80% identity to SEQ ID NO: 4 and the CYP450 protein (a) comprises an amino acid sequence having at least 85% identity to SEQ ID NO: 3; (b) comprises an amino acid sequence having at least 90% identity to SEQ ID NO: 3; (c) comprises an amino acid sequence having at least 95% identity to SEQ ID NO: 3; or (d) comprises the amino acid sequence of SEQ ID NO: 3.

16. The method of claim 13, wherein the nucleotide sequence has at least 80% identity to SEQ ID NO: 6 and the CYP450 protein (a) comprises an amino acid sequence having at least 85% identity to SEQ ID NO: 5; (b) comprises an amino acid sequence having at least 90% identity to SEQ ID NO: 5; (c) comprises an amino acid sequence having at least 95% identity to SEQ ID NO: 5; or (d) comprises the amino acid sequence of SEQ ID NO: 5.

17. The method of claim 12, wherein the C4-C20 carboxylic acid substrate comprises a C4-C20 carboxylic acid; a salt of a C4-C20 carboxylic acid; an ester of a C4-C20 carboxylic acid; a mono-, di- or triglyceride of a C4-C20 carboxylic acid; or a combination thereof.

18. The method of claim 12, wherein the C4-C20 carboxylic acid comprises a straight-chain C4-C20 carboxylic acid, a branched C4-C20 carboxylic acid, a saturated C4-C20 carboxylic acid, an unsaturated C4-C20 carboxylic acid, or a combination thereof.

19. The method of claim 14 for producing a C4-C20 gamma lactone selected from γ-heptalactone, γ-octalactone, γ-nonalactone, γ-decalactone, γ-undecalactone, γ-dodecalactone, γ-tridecalactone, and γ-tetradecalactone, wherein the C4-C20 carboxylic acid substrate is selected from heptanoic acid, octanoic acid, nonanoic acid, decanoic acid, undecanoic acid, dodecanoic acid, tridecanoic acid, and tetradecanoic acid.

20. The method of claim 15 for producing a C4-C20 gamma lactone selected from γ-undecalactone and γ-undecenolactone, wherein the C4-C20 carboxylic acid substrate is selected from undecanoic acid and undecenoic acid.

21. The method of claim 16 for producing a C4-C20 gamma lactone selected from γ-hexalactone, γ-heptalactone, γ-decalactone, γ-undecalactone, γ-dodecalactone, γ-tridecalactone, and γ-tetradecalactone, wherein the C4-C20 carboxylic acid substrate is selected from hexanoic acid, heptanoic acid, decanoic acid, undecanoic acid, dodecanoic acid, tridecanoic acid, and tetradecanoic acid.

22. The method of claim 12, wherein the transformed host cell is a growing cell or a resting cell.

23. (canceled)
